(19) United States
(12) Patent Application Publication
(10) Pub. No.: US 2011/0251286 A1
(43) Pub. Date: Oct. 13, 2011

(54) Crystalline salts and/or co-crystals of O-desmethyltramadol

(75) Inventors: Michael GRUSS, Aachen (DE); Oswald Zimmer, Wuerselen (DE)

(73) Assignee: GRUENENTHAL GmbH, Aachen (DE)

(21) Appl. No.: 13/044,069

(22) Filed: Mar. 9, 2011

(30) Foreign Application Priority Data
Mar. 10, 2010 (EP) 10 002 480.1

(51) Int. Cl. A61K 31/135; A61P 29/00; A61P 13/10; A61P 1/12; A61P 21/00; A61P 11/14; C07C 215/64; A61P 25/30

(52) U.S. Cl. 514/646; 554/121; 562/595; 554/1; 562/495; 554/171

(57) ABSTRACT

Salts and/or co-crystals of (+)-3-[2-(dimethylamino)-methyl-1-hydroxycyclohexyl]-phenol or of (−)-3-[2-(dimethylamino)methyl-1-hydroxy-cyclohexyl]phenol, present in crystalline form, with cinnamic acid, C₈- to C₁₀-alkane-monocarboxylic acids, C₆- to C₁₀-alkanedicarboxylic acids, C₁₅- to C₁₇-alkane-monocarboxylic acids, fumaric acid, tartaric acid, mandelic acid, hippuric acid and/or embonic acid, a process for their production, and the use of such salts and/or co-crystals as medicaments.

### CROSS REFERENCE TO RELATED APPLICATION

This application claims priority from European patent application no. EP 10002480.1, filed Mar. 10, 2010, the entire disclosure of which is incorporated herein by reference.

### BACKGROUND OF THE INVENTION

The present invention relates to salts and/or co-crystals of (+)-3-[2-(dimethylamino)-methyl-1-hydroxycyclohexyl]phenol or of (−)-3-[2-(dimethylamino)methyl-1-hydroxycyclohexyl]phenol, present in crystalline form, with aromatic or aliphatic carboxylic acids. It furthermore relates to a process for their production, salts and/or co-crystals of this type for use as medicaments, and their use for the production of medicaments.

One of the main metabolites of tramadol is 3-[2-(dimethylamino)methyl-1-hydroxycyclohexyl]phenol or O-desmethyltramadol (ODMT). This compound is pharmaceutically active itself. Its use as a free base and/or in the form of a physiologically tolerable salt for oral administration in the treatment of severe acute and/or chronic pain is described in EP 0 870 499 A1.

Crystalline forms of O-desmethyltramadol salts with carboxylic acids have not been described until now. Carboxylic acids are referred to for the formation of pharmaceutically tolerable salts. Thus EP 0 870 499 A1 discloses the use of salts of lipophilic organic acids, for example of C₈-C₂₂-fatty acids such as stearic acid and palmitic acid, with O-desmethyltramadol. Whether these are present in crystalline or amorphous form, however, is not mentioned.

The same situation exists in the patent application US 2004/0242617 A1 and in the patent specification U.S. Pat. No. 6,780,891, which mentions, inter alia, monomethylsebacic acid in a list as an acid forming pharmaceutically acceptable salts.

Crystalline forms of the active ingredient with carboxylic acids, however, would be desirable in order to impart to the active ingredient other pharmacological properties with, at the same time, good processability and better storage stability.

### SUMMARY OF THE INVENTION

According to the invention, salts and/or co-crystals of (+)-3-[2-(dimethylamino)-methyl-1-hydroxycyclohexyl]phenol or of (−)-3-[2-(dimethylamino)methyl-1-hydroxy-cyclohexyl]phenol, present in crystalline form, with aromatic or aliphatic carboxylic acids are therefore proposed, which are selected from the group comprising cinnamic acid, C₈- to C₁₀-alkanemonocarboxylic acids, C₈- to C₁₀-alkanedicarboxylic acids, C₁₅- to C₁₇-alkanemonocarboxylic acids, fumaric acid, tartaric acid, mandelic acid, hippuric acid and/or embonic acid.

Preferred C₆- to C₁₀-alkanemonocarboxylic acids are in this case hexanoic acid, octanoic acid and decanoic acid. A preferred C₈- to C₁₀-alkanedicarboxylic acid is sebacic acid (decanedicarboxylic acid). A preferred C₁₅- to C₁₇-alkanemonocarboxylic acid is palmitic acid.

According to the invention, additionally included in the definition of the crystalline forms of the salts and/or co-crystals are their hydrates, solvates and polymorphic forms.

The compounds according to the invention are present in crystalline form, the crystallinity being defined by the presence of at least one, preferably several, reflections in the X-ray powder diffractogram. It is also spoken in this connection of “X-ray crystalline”.

The crystalline state of a pharmaceutical active ingredient has several advantages compared to the amorphous form with respect to chemical stability, processability, purifiability during the preparation process and not least with respect to the possibility of the selection of forms such as anhydrates, hydrates and solvates.

The O-desmethyltramadol is employed either in the form of the (+)-enantiomer or of the (−)-enantiomer. This corresponds to a (1R,2R)- or a (1S,2S)-configuration. The (1R,2R)-configured (+)-enantiomer is preferred here, as is described by the formula (I):

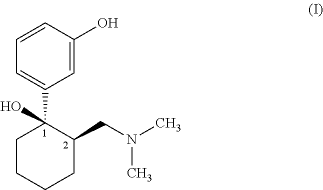

By reaction with the carboxylic acids mentioned, salts and/or co-crystals are obtained. The transition between salts and co-crystals is fluid here and is in the end determined by means of the position of the acidic proton of the carboxylic acid in the solid state. If the proton is closer to the aminic nitrogen atom, a salt is present. If it is closer to the carboxyl group, it is more appropriate to speak of a co-crystal.

The neutral compound 3-[2-(dimethylamino)methyl-1-hydroxycyclohexyl]phenol (“free base”) can be obtained from the commercially obtainable hydrochloride by reaction with a suitable base. Preferably, an aqueous solution of the hydrochloride is reacted with sodium hydroxide solution and the solid obtained is extracted with ethyl acetate.

The hydrochloride as such can also be obtained from the free base by reaction with hydrochloric acid. Thus, for example, a solution of the free base in acetone can be treated with concentrated hydrochloric acid (32%). Preferred forms of the hydrochloride are (1R,2R)- or (1S,2S)-configured and can furthermore independently thereof have a melting point determined by means of dynamic difference calorimetry (differential scanning calorimetry, DSC) of ≧245° C. to ≦253° C. or of ≧250° C. to ≦252° C. These are the peak temperatures in the DSC curve. Furthermore, the onset temperatures in the DSC curve can be in a range from ≧243° C. to ≦247° C. or from ≧244° C. to ≦246° C.

It is furthermore possible that the hydrochloride in the powder diffractogram, measured using CuK_{α} radiation, comprises one or more of the following reflections (in each case ±0.2 in 20): 11.1; 14.0; 16.7; 19.2; 19.5; 20.1; 20.3; 21.1; 21.8; 23.9; 25.7; 27.9; 30.9.

Preferably, the powder diffractogram comprises one or more of the following reflections (in each case ±0.2 in 2θ and measured using CuK_{α} radiation, the relative intensity being at most 100):

TABLE 1
Powder diffractogram

| 2θ | I(rel) |
|---|---|
| 11.07 | 60 |
| 13.95 | 26 |
| 16.96 | 100 |
| 19.22 | 26 |
| 19.53 | 97 |
| 20.09 | 38 |
| 20.29 | 66 |
| 21.09 | 25 |
| 21.83 | 26 |
| 23.87 | 60 |
| 25.73 | 42 |
| 27.94 | 22 |
| 30.89 | 40 |

Preferred forms of the free base are (1R,2R)- or (1S,2S)-configured and can furthermore independently thereof have a melting point of ≧138° C. to ≦143° C. or of ≧139° C. to ≦142° C. determined by means of dynamic difference calorimetry (differential scanning calorimetry, DSC). These are the peak temperatures in the DSC curve. Furthermore, the onset temperatures in the DSC curve can be in a range from ≧137° C. to ≦141° C. or from ≧138° C. to ≦140° C.

With respect to the free base, it is furthermore possible that the free base in the powder diffractogram, measured using CuK_{α} radiation, comprises one or more of the following reflections (in each case ±0.2 in 2θ): 11.7; 13.1; 14.4; 15.4; 17.8; 20.2; 20.5; 21.9; 22.7; 27.0; 28.2; 28.9.

Preferably, the powder diffractogram comprises one or more of the following reflections (in each case ±0.2 in 2θ and measured using CuK_{α} radiation, the relative intensity being at most 100):

TABLE 2
Powder diffractogram

| 2θ | I(rel) |
|---|---|
| 11.68 | 29 |
| 13.14 | 16 |
| 14.38 | 68 |
| 15.44 | 43 |
| 17.79 | 100 |
| 20.23 | 35 |
| 20.47 | 17 |
| 21.90 | 25 |
| 22.68 | 17 |
| 22.86 | 19 |
| 27.01 | 18 |
| 28.15 | 12 |
| 28.90 | 11 |

It is furthermore possible that the free base in the powder diffractogram, measured using CuK_{α} radiation, comprises one or more of the following reflections (in each case ±0.2 in 2θ): 14.3; 15.4; 17.7; 20.2; 21.8; 26.9.

Preferably, the powder diffractogram then comprises one or more of the following reflections (in each case ±0.2 in 2θ and measured using CuK_{α} radiation, the relative intensity being at most 100):

TABLE 3
Powder diffractogram

| 2θ | I(rel) |
|---|---|
| 14.29 | 34 |
| 15.35 | 32 |
| 17.73 | 100 |
| 20.18 | 25 |
| 21.84 | 20 |
| 26.93 | 16 |

The preferred forms of the free base mentioned are independently of one another already suitable for use as medicaments. In particular, this can be a medicament for the treatment of the following indications: pain, acute pain, chronic pain, neuropathic pain, visceral pain; migraine; depression; cough; urinary incontinence; irritable bladder; diarrhoea; pruritus; muscle spasms; cramps or convulsions; alcohol abuse, drug abuse, nicotine abuse, cocaine abuse; alcohol dependence, drug dependence, nicotine dependence, and/or cocaine dependence. Likewise, the preferred forms of the free base mentioned can independently of one another be used for the production of such medicaments.

In one embodiment of the salts and/or co-crystals according to the invention, the carboxylic acid is cinnamic acid and its powder diffractogram (of the salts and/or co-crystals) comprises one or more of the following reflections: (in each case ±0.2 in 2θ): 7.9; 11.4; 12.6; 14.7; 15.8; 16.1; 17.3; 18.7; 19.1; 19.9; 20.5; 21.3; 22.5; 23.4; 23.6; 29.7; 30.9. The reflections can be determined using CuK_{α} radiation.

These compounds can have a melting point of ≧182° C. to ≦186° C. or of ≧183° C. to ≦185° C. determined by means of DSC. These are the peak temperatures in the DSC curve. Furthermore, the onset temperatures in the DSC curve can be in a range from ≧181° C. to ≦185° C. or from ≧182° C. to ≦184° C.

Preferably, the powder diffractogram comprises one or more of the following reflections (in each case ±0.2 in 2θ and measured using CuK_{α} radiation, the relative intensity being at most 100):

TABLE 4
Powder diffractogram

| 2θ | I(rel) |
|---|---|
| 7.88 | 41 |
| 11.44 | 22 |
| 12.64 | 16 |
| 14.68 | 18 |
| 15.79 | 24 |
| 16.08 | 88 |
| 17.29 | 32 |
| 18.65 | 11 |
| 19.08 | 49 |
| 19.90 | 100 |
| 20.55 | 31 |
| 21.33 | 31 |
| 22.54 | 31 |
| 23.56 | 28 |
| 29.70 | 16 |
| 30.95 | 20 |

In a further embodiment of the salts and/or co-crystals according to the invention, the carboxylic acid is palmitic acid and its powder diffractogram (of the salts and/or co-crystals) comprises one or more of the following reflections: (in each case ±0.2 in 2θ): 5.3; 7.9; 8.7; 10.4; 11.6; 13.0; 13.5; 15.8; 16.5; 17.4; 17.7; 19.0; 20.1; 20.3; 20.5; 21.3; 22.1; 22.5; 23.2; 24.0. The reflections can be determined using CuK_{α} radiation.

These compounds can have a melting point of ≧76° C. to ≦84° C. or of ≧81° C. to ≦83° C. determined by means of DSC. These are the peak temperatures in the DSC curve. Furthermore, the onset temperatures in the DSC curve can be in a range from ≧76° C. to ≦80° C. or from ≧77° C. to ≦79° C.

Preferably, the powder diffractogram comprises one or more of the following reflections (in each case ±0.2 in 2θ and measured using CuKₐ radiation, the relative intensity being at most 100):

TABLE 5
Powder diffractogram

| 2θ | I(rel) |
|---|---|
| 5.31 | 100 |
| 7.94 | 18 |
| 8.69 | 11 |
| 10.35 | 16 |
| 11.56 | 30 |
| 13.00 | 22 |
| 13.47 | 12 |
| 15.79 | 37 |
| 16.45 | 33 |
| 17.40 | 70 |
| 17.75 | 18 |
| 18.98 | 56 |
| 20.07 | 14 |
| 20.35 | 39 |
| 20.53 | 48 |
| 21.30 | 25 |
| 22.11 | 20 |
| 22.47 | 14 |
| 23.17 | 20 |
| 23.97 | 25 |

In a further embodiment of the salts and/or co-crystals according to the invention, the carboxylic acid is octanoic acid and its powder diffractogram (of the salts and/or co-crystals) comprises one or more of the following reflections: (in each case ±0.2 in 2θ): 7.6; 12.9; 16.6; 18.5; 18.8; 19.6; 20.8; 22.2; 23.7; 24.0; 31.9. The reflections can be determined using CuK_{α} radiation.

These compounds can have a melting point of ≧100° C. to ≦104° C. or of ≧101° C. to ≦103° C. determined by means of DSC. These are the peak temperatures in the DSC curve. Furthermore, the onset temperatures in the DSC curve can be in a range from ≧98° C. to ≦102° C. or from a ≧99° C. to ≦101° C.

Preferably, the powder diffractogram comprises one or more of the following reflections (in each case ±0.2 in 2θ and measured using CuK_{α} radiation, the relative intensity being at most 100):

TABLE 6
Powder diffractogram

| 2θ | I(rel) |
|---|---|
| 7.63 | 23 |
| 12.87 | 28 |
| 16.59 | 100 |
| 18.50 | 27 |
| 18.77 | 45 |
| 19.60 | 68 |
| 20.81 | 65 |
| 22.18 | 40 |
| 23.30 | 10 |
| 23.70 | 15 |
| 24.01 | 13 |
| 31.88 | 20 |

In a further embodiment of the salts and/or co-crystals according to the invention, the carboxylic acid is decanoic acid and its powder diffractogram (of the salts and/or co-crystals) comprises one or more of the following reflections: (in each case ±0.2 in 2θ): 5.4; 6.1; 7.1; 8.3; 10.3; 12.6; 13.3; 14.2; 15.9; 16.8; 17.6; 18.7; 19.0; 19.8; 20.2; 20.4; 20.9; 21.5; 21.7; 22.5; 23.1; 23.8; 31.9. The reflections can be determined using CuK_{α} radiation.

These compounds can have a melting point of a ≧95° C. to ≦99° C. or of a ≧97° C. to ≦98° C. determined by means of DSC. These are the peak temperatures in the DSC curve. Furthermore, the onset temperatures in the DSC curve can be in a range from ≧93° C. to ≦97° C. or from a ≧94° C. to ≦96° C.

Preferably, the powder diffractogram comprises one or more of the following reflections (in each case ±0.2 in 2θ and measured using CuK_{α} radiation, the relative intensity being at most 100):

TABLE 7
Powder diffractogram

| 2θ | I(rel) |
|---|---|
| 5.42 | 80 |
| 6.12 | 100 |
| 7.13 | 26 |
| 8.34 | 19 |
| 10.34 | 14 |
| 12.60 | 27 |
| 13.32 | 22 |
| 14.21 | 17 |
| 15.88 | 68 |
| 16.80 | 22 |
| 17.63 | 30 |
| 18.73 | 36 |
| 19.01 | 33 |
| 19.83 | 26 |
| 20.15 | 22 |
| 20.44 | 11 |
| 20.86 | 40 |
| 21.50 | 12 |
| 21.75 | 11 |
| 22.48 | 11 |
| 23.06 | 13 |
| 23.83 | 12 |
| 31.94 | 11 |

In a further embodiment of the salts and/or co-crystals according to the invention, the carboxylic acid is fumaric acid and its powder diffractogram (of the salts and/or co-crystals) comprises one or more of the following reflections: (in each case ±0.2 in 2θ): 7.2; 9.4; 9.5; 9.9; 13.1; 13.5; 13.9; 14.3; 14.5; 15.3; 17.8; 18.0; 18.9; 19.2; 19.9; 20.1; 20.4; 20.7; 21.2; 21.8; 22.3; 22.8; 23.3; 25.3; 25.9; 26.1; 27.4; 28.0; 28.8; 35.7. The reflections can be determined using CuK_{α} radiation.

These compounds, inter alia, can have a melting point of ≧93° C. to ≦99° C. or of ≧96° C. to ≦98° C. determined by means of DSC. These are the peak temperatures in the DSC curve. Furthermore, the onset temperatures in the DSC curve can, inter alia, be in a range from ≧91° C. to ≦95° C. or from ≧92° C. to ≦94° C.

Preferably, the powder diffractogram comprises one or more of the following reflections (in each case ±0.2 in 2θ and measured using CuK_{α} radiation, the relative intensity being at most 100):

TABLE 8
Powder diffractogram

| 2θ | I(rel) |
|---|---|
| 7.16 | 46 |
| 9.43 | 31 |
| 9.54 | 24 |
| 9.91 | 26 |
| 13.06 | 100 |
| 13.51 | 29 |
| 13.92 | 23 |
| 14.33 | 56 |
| 14.50 | 21 |
| 15.30 | 14 |
| 17.76 | 13 |
| 18.01 | 53 |
| 18.89 | 32 |
| 19.19 | 42 |
| 19.88 | 12 |
| 20.12 | 20 |
| 20.42 | 96 |
| 20.65 | 14 |
| 21.22 | 20 |
| 21.80 | 75 |
| 22.31 | 14 |
| 22.76 | 26 |
| 23.29 | 13 |
| 25.34 | 11 |
| 25.90 | 12 |
| 26.08 | 11 |
| 27.13 | 6 |
| 27.39 | 13 |
| 28.01 | 15 |
| 28.84 | 18 |
| 35.66 | 13 |

In a further embodiment of the salts and/or co-crystals according to the invention, the carboxylic acid is sebacic acid and its powder diffractogram (of the salts and/or co-crystals) comprises one or more of the following reflections: (in each case ±0.2 in 2θ): 7.9; 8.6; 11.7; 12.6; 13.1; 13.5; 14.2; 15.8; 16.4; 16.7; 17.1; 18.0; 18.5; 19.0; 19.5; 20.2; 20.6; 21.5; 22.5; 22.9; 23.5; 24.2; 24.6; 25.8; 26.1; 26.4; 27.5; 32.2; 32.4. The reflections can be determined using CuK_{α} radiation.

These compounds can have a melting point of ≧158° C. to ≦162° C. or of ≧159° C. to ≦161° C. determined by means of DSC. These are the peak temperatures in the DSC curve. Furthermore, the onset temperatures in the DSC curve can be in a range from ≧157° C. to ≦161° C. or from ≧158° C. to ≦160° C.

Preferably, the powder diffractogram comprises one or more of the following reflections (in each case ±0.2 in 2θ and measured using CuK_{α} radiation, the relative intensity being at most 100):

TABLE 9
Powder diffractogram

| 2θ | I(rel) |
|---|---|
| 7.87 | 45 |
| 8.61 | 40 |
| 11.70 | 22 |
| 12.59 | 41 |
| 13.13 | 33 |
| 13.53 | 82 |
| 14.15 | 62 |
| 15.77 | 48 |
| 16.39 | 30 |
| 16.71 | 12 |
| 17.15 | 27 |
| 18.04 | 13 |
| 18.47 | 60 |
| 19.00 | 41 |
| 19.47 | 44 |
| 20.23 | 14 |
| 20.63 | 81 |
| 21.47 | 32 |
| 22.52 | 25 |
| 22.94 | 100 |
| 23.50 | 24 |
| 24.18 | 14 |
| 24.61 | 28 |
| 25.78 | 12 |
| 26.05 | 15 |
| 26.40 | 16 |
| 27.49 | 12 |
| 32.16 | 12 |
| 32.37 | 23 |

A further subject of the present invention is a process for the production of salts and/or co-crystals according to the invention, comprising the steps:
providing (+)-3-[2-(dimethylamino)methyl-1-hydroxycyclohexyl]phenol or of (−)-3-[2-(dimethylamino)methyl-1-hydroxycyclohexyl]phenol;
providing an aromatic or aliphatic carboxylic acid that is selected from the group comprising cinnamic acid, C₆- to C₁₀-alkanemonocarboxylic acids, C₈- to C₁₀-alkanedicarboxylic acids, C₁₅- to C₁₇-alkanemonocarboxylic acids, fumaric acid, tartaric acid, mandelic acid, hippuric acid and/or embonic acid;
addition of a suitable solvent to the mixture of 3-[2-(dimethylamino)methyl-1-hydroxycyclohexyl]phenol and the carboxylic acid obtained; and
removal of the solvent.

Preferably, stoichiometric amounts of 3-[2-(dimethylamino)methyl-1-hydroxycyclohexyl]phenol and of the carboxylic acid are employed. By this, it is in particular also to be understood that one mole of carboxyl groups of the carboxylic acid reacts with one mole of 3-[2-(dimethylamino)methyl-1-hydroxycyclohexyl]phenol.

Additionally included here are also technically unavoidable variations of the proportions on account of inaccuracies of metering, etc. Furthermore, this definition also comprises ratios between 3-[2-(dimethylamino)methyl-1-hydroxycyclohexyl]-phenol and the carboxylic acid that are established by the formation of mixed crystals.

Preferred carboxylic acids were already mentioned in connection with the salts and/or co-crystals, so reference is made thereto.

In principle, the sequence of the preparation of the reaction components and the addition of the solvent is not fixed. It is likewise conceivable that solutions of the free base and of the carboxylic acid are combined. Advantageously, a procedure can be used such that 3-[2-(dimethylamino)methyl-1-hydroxycyclohexyl]phenol and the carboxylic acid are introduced into the reaction vessel together and the solvent is added at room temperature. The reaction period can be, for example, ≧4 hours to ≦24 hours. After this the solvent is removed, for example by evaporating or introducing an inert gas. The desired product is thereby obtained.

Suitable solvents, depending on the acid employed, are in particular ethers such as tert-butyl methyl ether, acetone, halogenated hydrocarbons, ethyl acetate and isopropanol.

In one embodiment of the process according to the invention, the carboxylic acid is not sebacic acid and the solvent is dichloromethane. In particular, the carboxylic acid can be cinnamic acid, fumaric acid, octanoic acid, decanoic acid or palmitic acid. With the exception mentioned, dichloromethane then leads to solid reaction products after evaporation.

In a further embodiment of the process according to the invention, the carboxylic acid is sebacic acid and the solvent is tert-butyl methyl ether, acetone or isopropanol.

The present invention furthermore relates to salts and/or co-crystals according to the invention for use as medicaments.

Preferably, these are salts and/or co-crystals according to the invention for use as medicaments, where the medicament is a medicament having delayed, prolonged and/or extended release.

According to the invention, the term “delayed release” means that the active ingredient is released over a longer period of time with the aim of a lengthened therapeutic action in comparison to undelayed release. A “prolonged release” takes place continuously over a longer period of time, but where the rate of release decreases with time. Finally, “extended release” is spoken of if the elimination phase is superimposed until the conclusion of the slow release and an apparent lengthening of the elimination half-life occurs. A summary of these terms is also found in the “Lehrbuch der Pharmazeutischen Technologie” [Textbook of Pharmaceutical Technology], K. H. Bauer et al., Wissenschaftliche Verlagsgesellschaft Stuttgart; 6th Edition (1999), Chapter 16.

A delayed, prolonged and/or extended release can be achieved, for example by employing matrix forming agents such as cellulose ethers in the pharmaceutical formulation or using enteric-coated preparations. In the case of salts and/or co-crystals according to the invention with decreased release rates compared to the hydrochloride salt, for example, in artificial intestinal juice, the substance properties as such can already provide for a delayed release. These salts and/or co-crystals are the cinnamates, palmitates, decanoates, octanoates and hemi-sebacates.

The medicament is suitable for administration to adults and children including infants and babies and can preferably be present as a solid, but also as a liquid or semisolid pharmaceutical form, for example in the form of injection solutions, drops, juices, syrups, sprays, suspensions, tablets, patches, capsules, suppositories, ointments, creams, lotions, gels, emulsions, aerosols or in multi-particulate form, for example in the form of pellets or granules, optionally compressed to give tablets, filled into capsules or suspended in a liquid, and also administered as such.

Additionally, the medicament can customarily contain further physiologically tolerable pharmaceutical excipients, which can be selected, for example, from the group consisting of carrier materials, fillers, solvents, diluents, surface-active substances, colorants, preservatives, disintegrants, glidants, lubricants, flavourings and binders.

The selection of the physiologically tolerable excipients and the amounts thereof to be employed depends on whether the medicament is to be administered orally, subcutaneously, parenterally, intravenously, intraperitoneally, intradermally, intramuscularly, intranasally, buccally, rectally or locally, for example to infections on the skin, of the mucous membranes and to the eyes. For oral administration, suitable preparations are preferably those in the form of tablets, coated tablets, capsules, granules, pellets, drops, juices and syrups, for parenteral administration, topical and inhalative administration solutions, suspensions, easily reconstitutable dry preparations, and sprays. The salts and/or co-crystals, in particular the palmitates, used in the medicament in a depot in dissolved form or in a patch, optionally with addition of agents promoting skin penetration, are suitable preparations for percutaneous application. Orally or percutaneously administrable preparation forms can also release the salts and/or co-crystals with a delay.

The medicaments are produced with the aid of customary agents, devices, methods and processes known from the prior art, as are described, for example, in “Remington's Pharmaceutical Sciences”, editor A. R. Gennaro, 17th Edition, Mack Publishing Company, Easton, Pa., 1985, in particular in Part 8, Chapters 76 to 93. The corresponding description is hereby inserted as a reference and is to be considered as part of the disclosure. The amount of the respective salts and/or co-crystals to be administered to the patient can vary and is dependent, for example, on the weight or age of the patient and on the type of administration, the indication and the degree of severity of the disease. Customarily, 0.001 to 100 mg/kg, preferably 0.05 to 75 mg/kg, particularly preferably 0.05 to 50 mg/kg, of body weight of the patient are administered.

Furthermore, the present invention relates to salts and/or co-crystals according to the invention for the treatment and/or prophylaxis of one or more diseases or conditions selected from the group comprising:
pain, acute pain, chronic pain, neuropathic pain, visceral pain;
migraine; depression;
cough; urinary incontinence; irritable bladder; diarrhoea; pruritus; muscle spasms; cramps or convulsions;
alcohol abuse, drug abuse, nicotine abuse, cocaine abuse;
alcohol dependence, drug dependence, nicotine dependence, and/or cocaine dependence.

The present invention furthermore relates to the use of salts and/or co-crystals according to the invention for production of a medicament for the treatment and/or prophylaxis of one or more diseases or conditions selected from the group comprising:
pain, acute pain, chronic pain, neuropathic pain, visceral pain;
migraine; depression;
cough; urinary incontinence; irritable bladder; diarrhoea; pruritus; muscle spasms; cramps or convulsions;
alcohol abuse, drug abuse, nicotine abuse, cocaine abuse;
alcohol dependence, drug dependence, nicotine dependence, and/or cocaine dependence.

Preferably, the use here is one where the medicament is a medicament having delayed, prolonged and/or extended release. These terms have already been explained above.

A further subject of the invention is a process for the treatment, in particular in one of the aforementioned indications, of a nonhuman mammal or human that needs treatment of the corresponding indication by administration of a therapeutically efficacious dose of a medicament according to the invention.

### BRIEF DESCRIPTION OF THE DRAWINGS

The invention will be described in further detail hereinafter with reference to illustrative examples for which X-ray diffraction spectra are shown in the accompanying drawings.

FIG. 1a shows an X-ray powder diffractogram spectrum of the free base of Example 1a.

FIG. 1b shows a further X-ray powder diffractogram spectrum of the free base of Example 1b.

FIG. 1c shows an X-ray powder diffractogram spectrum of the hydrochloride

FIG. 2 shows a structural element of the monocrystal structure of the cinnamate.

FIG. 3 shows an X-ray powder diffractogram spectrum of the cinnamate.

FIG. 4 shows an X-ray powder diffractogram spectrum of the palmitate.

FIG. 5a shows an X-ray powder diffractogram spectrum of the octanoate.

FIG. 5b shows a further X-ray powder diffractogram spectrum of the octanoate.

FIG. 5c shows a further X-ray powder diffractogram spectrum of the octanoate.

FIG. 6 shows an X-ray powder diffractogram spectrum of the decanoate.

FIG. 7 shows an X-ray powder diffractogram spectrum of the hemi-fumarate.

FIG. 8 shows an X-ray powder diffractogram spectrum of the hemi-sebacate.

FIG. 9 shows a dynamic vapor sorption spectrum of the free base.

FIG. 10 shows a dynamic vapor sorption spectrum of the cinnamate.

FIG. 11 shows a dynamic vapor sorption spectrum of the palmitate.

FIG. 12 shows a dynamic vapor sorption spectrum of the hemi-fumarate.

FIG. 13 shows a dynamic vapor sorption spectrum of the hemi-sebacate.

### DETAILED DESCRIPTION OF EXAMPLES

The present invention is further illustrated by the following examples, but without being restricted thereto.

### Apparatus and Methods:

Differential scanning calorimetry (DSC): apparatuses of the type Mettler Toledo DSC821e were used. Measurements were generally carried out in aluminium crucibles. If not stated otherwise, the heating rate was 10° C. per minute, and amounts of substance between 2 and 20 mg were employed.

X-ray powder diffractometry (XRPD): XRP diffractograms were carried out using a Stoe Stadi P X-ray powder diffractometer, CuK_{α} radiation being used. D distances were calculated from the 2θ values, the wavelength of 1.54060 Å being used as a basis. It generally applies that the 2θ values have an error rate of ±0.1° to ±0.2°. The experimental error in the case of the D distance values is therefore dependent on the location of the line (of the reflection or peak).

Monocrystal structural investigations were carried out on a Bruker D8 goniometer with a SMART APEX CCD surface detector at 100 K using MoK_{α} radiation (λ=0.71073 Å, graphite monochromator).

Investigations on the sorption and desorption of moisture (dynamic vapor sorption, DVS): DVS measurements were carried out on an apparatus of the type DVS 1000 from Porotec. The evaluation of the measurements was carried out using software from Surface Measurement Systems. At a temperature between 23° C. and 26° C., starting from a relative humidity of 50%, within 240 minutes the relative humidity first increased to 90% and then fell to 0%. This cycle was repeated a second time. The DVS charts shown are isothermal recordings (plots) of the change in the mass in % (change in mass—dry) as a function of the desired relative humidity in % (target RH). Correspondingly, the axes of the graphs are marked by ΔM [%] and RH [%]. The measurement curves of the first sorption are designated by 1-S, the subsequent first desorption by 1-D, the second sorption by 2-S and the second desorption by 2-D. Advantageously, mass changes between the sorption and desorption phase are as low as possible.

Investigations on release were carried out in a Sotax AT7 smart release apparatus with UV measurement (Perkin Elmer photometer).

A vessel with Wood apparatus was used (for intrinsic release).

A pressing of 0.5 cm² surface area (diameter 8 mm) was produced with a force of 200 N using approximately 100 mg of active ingredient.

Release was carried out in a release medium, which is composed as described below:
Intestinal juice without pancreas powder, pH 6.8 (analogous to Ph. Eur.); 250.0 ml 6.8 g of potassium dihydrogenphosphate R+0.896 g of NaOH+water to 1000.0 ml.

Release was carried out in a release volume of in each case 500±5 ml at a temperature of the release medium of 37±1° C. with a stirring speed of 100±5 rpm.

The content determination was carried out by means of spectroscopic methods (UV spectroscopy, wavelength: 271 nm, cuvette: 10 mm). Sample taking was carried out manually.

In the following examples, the formation of the compounds with cinnamic acid, palmitic acid, octanoic acid, decanoic acid, fumaric acid and sebacic acid is described, but without making a statement by means of the word choice about whether they are salts or co-crystals.

The neutral compound 3-[2-(dimethylamino)methyl-1-hydroxycyclohexyl]phenol (“free base”) can be obtained from the commercially obtainable hydrochloride by reaction with sodium hydroxide solution by means of customary methods, as is also described below.

Generally, the procedure was such that firstly the free base and the corresponding acid were introduced in solid form and the solvent was then added. In the case of liquid acids, the base was initially introduced, then the solvent and subsequently the acid was added.

The salts and/or co-crystals obtained in the examples were investigated by means of ¹H-NMR spectroscopy. Indications of decomposition or the formation of other compounds were not found in any ¹H-NMR spectrum.

### Example 1

### Release of the Base

1a:

10.19 g of (+)-(1R,2R)-3-[2-(dimethylamino)methyl-1-hydroxycyclohexyl]phenol hydrochloride were introduced into 250 ml of dist. water in a three-necked flask. A clear solution of pH 6.0 was obtained. In the course of 24 minutes, 36.7 ml of sodium hydroxide solution (c=1 mol/l) were added dropwise at 22° C. and a stirring speed of 600 rpm. The pH of the reaction mixture was monitored. The appearance of a white solid at the site of entry of the sodium hydroxide solution was observed, which went into solution again up to a pH of 9.1. After the addition of 5 ml of sodium hydroxide solution, the pH was 8.3 and after 10 ml 8.83. From a pH of 9.1, turbidity of the reaction mixture was visible and from pH 9.3 a white, flocculent precipitate deposited. After the end of the addition of sodium hydroxide solution, the pH was 9.93. The mixture was subsequently stirred at 22° C. for 15 minutes.

For the work-up of the suspension obtained, 200 ml of ethyl acetate were added to dissolve the precipitated solid. The organic phase was separated off in a separating funnel and the aqueous phase was extracted a further two times with 100 ml each of ethyl acetate. The collected organic phases were dried over magnesium sulfate for 10 minutes. After filtration, the organic phase was freed from the solvent in vacuo until dry (50° C., 3 hours).

8.6 g (96.7%) of a white solid were obtained.

Melting point (DSC): 139.7° C. (onset); 141.3 (peak); 110.4 J/g

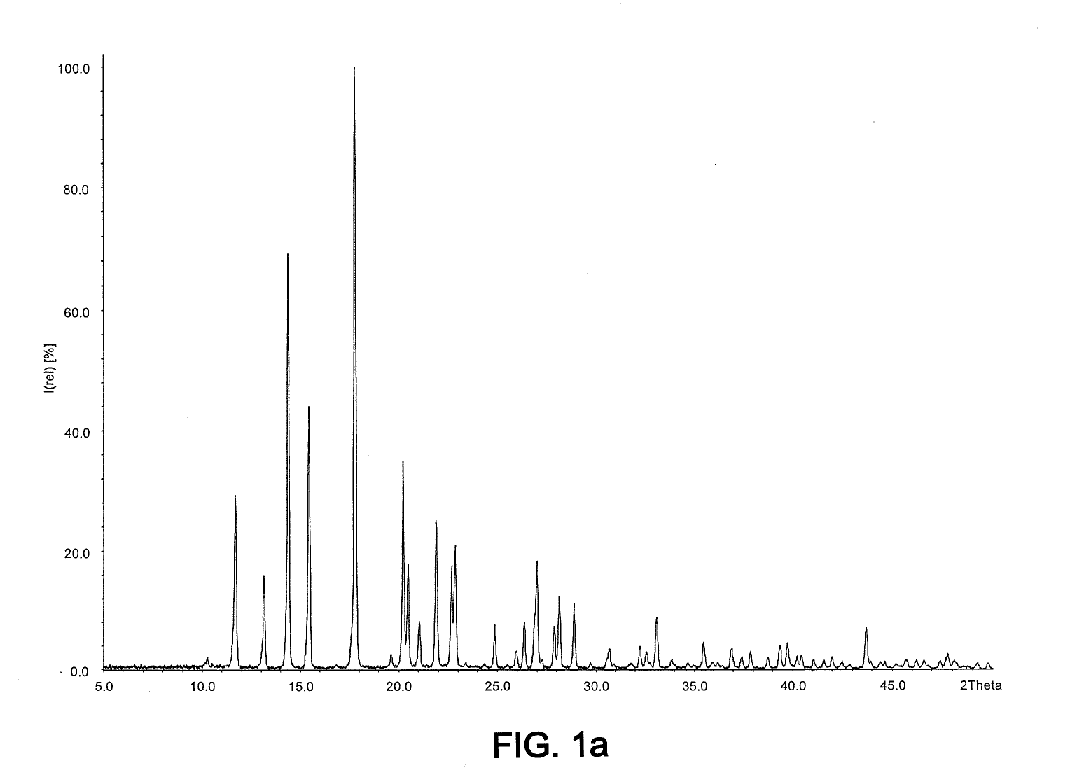

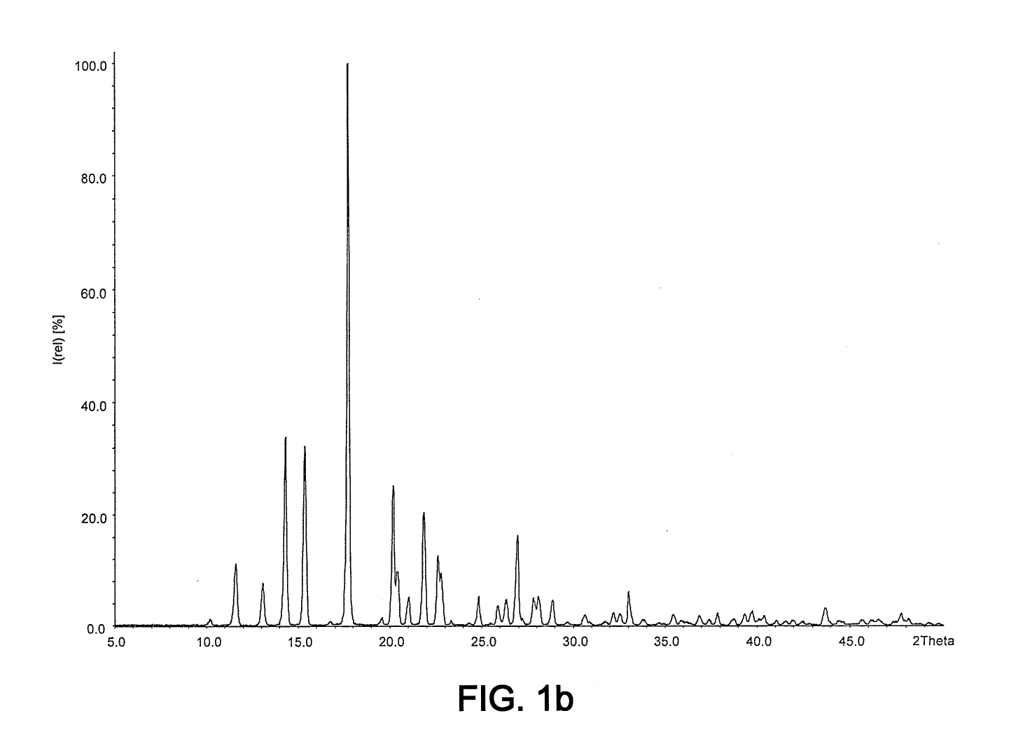

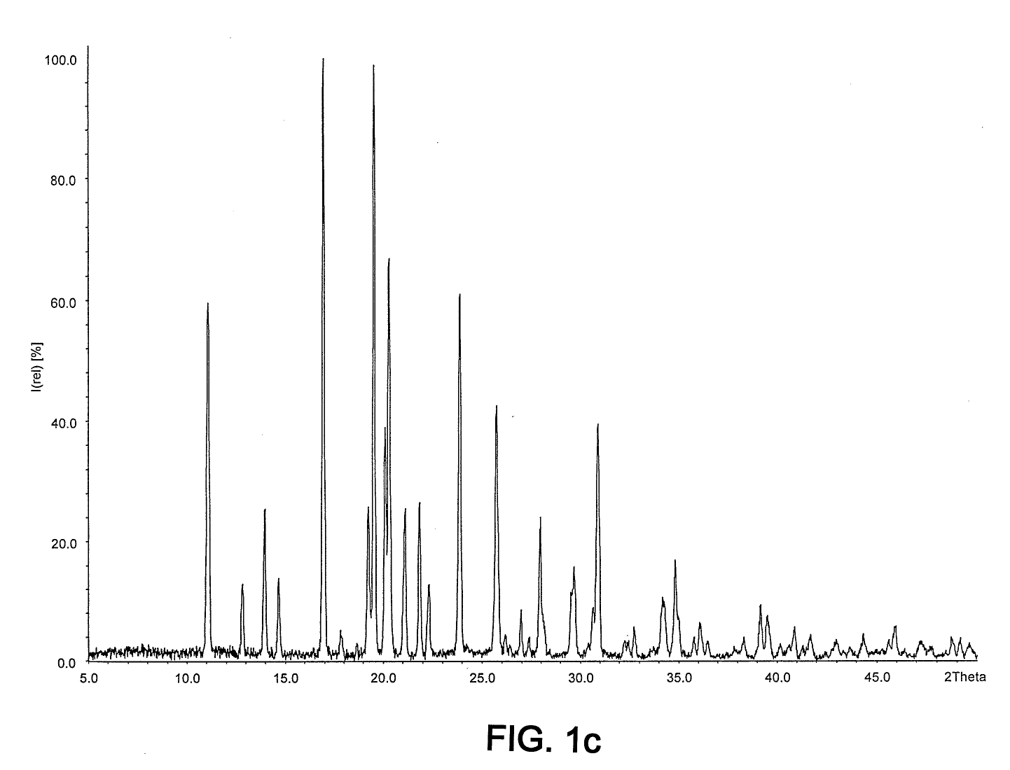

XRPD table of the product from this batch (shown graphically in FIG. 1a):

TABLE 10
Powder diffractogram

| D | 2θ | I(rel) |
|---|---|---|
| 8.63 | 10.25 | 2 |
| 7.57 | 11.68 | 29 |
| 6.73 | 13.14 | 16 |
| 6.16 | 14.38 | 68 |
| 5.74 | 15.44 | 43 |
| 5.27 | 16.81 | 1 |
| 4.98 | 17.79 | 100 |
| 4.52 | 19.62 | 2 |
| 4.39 | 20.23 | 35 |
| 4.33 | 20.47 | 17 |
| 4.22 | 21.04 | 8 |
| 4.05 | 21.90 | 25 |
| 3.92 | 22.68 | 17 |
| 3.89 | 22.86 | 19 |
| 3.80 | 23.38 | 1 |
| 3.65 | 24.35 | 1 |
| 3.58 | 24.86 | 8 |
| 3.43 | 25.96 | 3 |
| 3.38 | 26.37 | 8 |
| 3.30 | 27.01 | 18 |
| 3.27 | 27.27 | 2 |
| 3.20 | 27.89 | 7 |
| 3.17 | 28.15 | 12 |
| 3.09 | 28.90 | 11 |
| 3.00 | 29.72 | 1 |
| 2.91 | 30.69 | 3 |
| 2.89 | 30.90 | 1 |
| 2.87 | 31.10 | 0 |
| 2.81 | 31.79 | 1 |
| 2.77 | 32.24 | 4 |
| 2.75 | 32.56 | 3 |
| 2.73 | 32.72 | 1 |
| 2.71 | 33.08 | 9 |
| 2.65 | 33.85 | 1 |
| 2.59 | 34.67 | 1 |
| 2.56 | 34.99 | 1 |
| 2.53 | 35.48 | 4 |
| 2.50 | 35.94 | 1 |
| 2.48 | 36.20 | 1 |
| 2.46 | 36.43 | 1 |
| 2.43 | 36.90 | 3 |
| 2.40 | 37.42 | 2 |
| 2.37 | 37.87 | 3 |
| 2.32 | 38.74 | 2 |
| 2.29 | 39.36 | 4 |
| 2.27 | 39.74 | 4 |

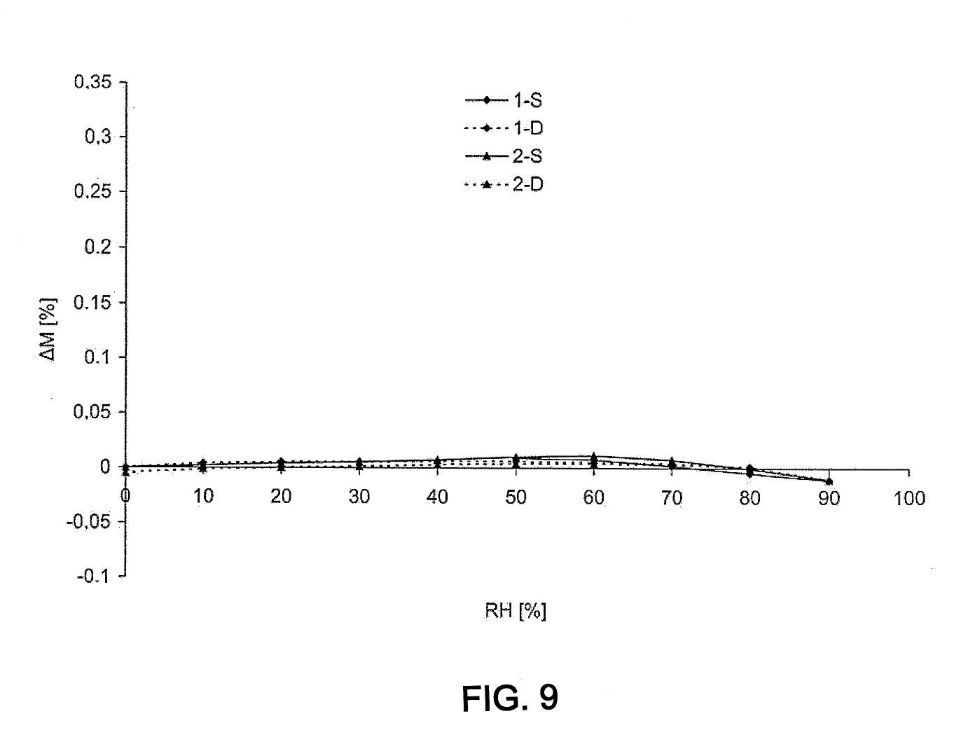

The dynamic vapor sorption spectrum of the free base is reproduced in FIG. 9. The measurements are found in the following table. A third partial cycle not shown in the figure is additionally presented.

TABLE 11
DVS data

ΔM [%]

| | RH [%] | Sorption | Desorption | Hysteresis |
|---|---|---|---|---|
| 1st cycle | 0.0 | | 0.00000 | |
| | 10.0 | | 0.00395 | |
| | 20.0 | | 0.00526 | |
| | 30.0 | | 0.00526 | |
| | 40.0 | | 0.00636 | |
| | 50.0 | 0.00855 | 0.00614 | −0.00241 |
| | 60.0 | 0.00746 | 0.00526 | −0.00219 |
| | 70.0 | 0.00175 | 0.00395 | 0.00219 |
| | 80.0 | −0.00461 | 0.00132 | 0.00592 |
| | 90.0 | −0.01097 | −0.01097 | |
| 2nd cycle | 0.0 | 0.00000 | −0.00461 | |
| | 10.0 | 0.00197 | −0.00132 | −0.00329 |
| | 20.0 | 0.00395 | 0.00044 | −0.00351 |
| | 30.0 | 0.00526 | 0.00132 | −0.00395 |
| | 40.0 | 0.00702 | 0.00307 | −0.00395 |
| | 50.0 | 0.00987 | 0.00395 | −0.00592 |
| | 60.0 | 0.01119 | 0.00395 | −0.00724 |
| | 70.0 | 0.00746 | 0.00329 | −0.00417 |
| | 80.0 | −0.00066 | 0.00132 | 0.00197 |
| | 90.0 | −0.01031 | −0.01031 | |
| 3rd cycle | 0.0 | −0.00461 | | |
| | 10.0 | −0.00110 | | |
| | 20.0 | 0.00066 | | |
| | 30.0 | 0.00197 | | |
| | 40.0 | 0.00461 | | |
| | 50.0 | 0.00768 | | |

1b:

60.0 g of (+)-(1R,2R)-3-[2-(dimethylamino)methyl-1-hydroxycyclohexyl]phenol hydrochloride (209.9 mmol) were initially introduced into a 3-necked flask in 1.5 l of dist. water. 209.9 ml of sodium hydroxide solution (c=1 mol/l) were added dropwise at room temperature in the course of 30 minutes at a pH of 6.46. The pH of the reaction mixture was monitored. After the addition of 10 ml of sodium hydroxide solution, the pH was 8.27, after 50 ml 8.87 and after 210 ml 9.83. The precipitation of a white solid was observed. After 160 ml of added sodium hydroxide solution, 300 ml of ethyl acetate were added, since uniform stirring was no longer possible. After addition of the sodium hydroxide solution was complete, the reaction mixture was subsequently stirred for 15 minutes and 800 ml of ethyl acetate were again added to dissolve the solid completely.

For work-up, the solution was transferred to a separating funnel and the lower, aqueous phase was discharged. The aqueous phase was extracted with 2×600 ml of ethyl acetate and the organic phases collected were combined, dried over magnesium sulfate, filtered off and then concentrated to dryness in vacuo (50° C., 3 hours)

46.146 g (88.2%) of a white solid were obtained.

Melting point (DSC): 140.3° C. (onset); 144.2 (peak); 134.6 J/g

XRPD table of the product from this batch (shown graphically in FIG. 1b):

TABLE 12
Powder diffractogram

| D | 2θ | I(rel) |
|---|---|---|
| 8.69 | 10.17 | 1 |
| 7.64 | 11.58 | 11 |
| 6.78 | 13.05 | 7 |
| 6.19 | 14.29 | 34 |
| 5.77 | 15.35 | 32 |
| 5.29 | 16.73 | 1 |
| 5.00 | 17.73 | 100 |
| 4.54 | 19.54 | 1 |
| 4.40 | 20.18 | 25 |
| 4.35 | 20.40 | 10 |
| 4.23 | 21.00 | 5 |
| 4.07 | 21.84 | 20 |
| 3.93 | 22.60 | 12 |
| 3.90 | 22.77 | 9 |
| 3.81 | 23.32 | 1 |
| 3.66 | 24.30 | 0 |
| 3.58 | 24.82 | 5 |
| 3.49 | 25.48 | 0 |
| 3.44 | 25.87 | 3 |
| 3.38 | 26.32 | 4 |
| 3.31 | 26.93 | 16 |
| 3.27 | 27.21 | 1 |
| 3.21 | 27.81 | 5 |
| 3.17 | 28.08 | 5 |
| 3.09 | 28.85 | 4 |
| 3.01 | 29.66 | 1 |
| 2.92 | 30.61 | 2 |
| 2.82 | 31.72 | 1 |
| 2.78 | 32.16 | 2 |
| 2.75 | 32.52 | 2 |
| 2.71 | 33.00 | 6 |
| 2.65 | 33.79 | 1 |
| 2.59 | 34.65 | 0 |
| 2.53 | 35.44 | 2 |
| 2.50 | 35.87 | 1 |
| 2.48 | 36.22 | 0 |
| 2.44 | 36.86 | 2 |
| 2.40 | 37.39 | 1 |
| 2.38 | 37.85 | 2 |
| 2.32 | 38.73 | 1 |
| 2.29 | 39.33 | 2 |
| 2.27 | 39.73 | 2 |

An XRPD measurement, whose results are shown graphically in FIG. 1c, was likewise carried out on the starting substance (+)-(1R,2R)-3-[2-(dimethylamino)-methyl-1-hydroxycyclohexyl]phenol hydrochloride. The XRPD table appears as follows:

TABLE 13
Powder diffractogram

| D | 2θ | I(rel) |
|---|---|---|
| 7.99 | 11.07 | 60 |
| 6.90 | 12.82 | 13 |
| 6.34 | 13.95 | 26 |
| 6.04 | 14.66 | 14 |
| 5.22 | 16.96 | 100 |
| 4.97 | 17.84 | 4 |
| 4.76 | 18.64 | 3 |
| 4.61 | 19.22 | 26 |
| 4.54 | 19.53 | 97 |
| 4.42 | 20.09 | 38 |
| 4.37 | 20.29 | 66 |
| 4.21 | 21.09 | 25 |
| 4.07 | 21.83 | 26 |
| 3.98 | 22.29 | 13 |
| 3.72 | 23.87 | 60 |
| 3.46 | 25.73 | 42 |
| 3.40 | 26.18 | 4 |
| 3.37 | 26.40 | 2 |
| 3.30 | 26.96 | 8 |
| 3.25 | 27.40 | 4 |
| 3.19 | 27.94 | 22 |
| 3.02 | 29.52 | 11 |
| 3.01 | 29.67 | 15 |
| 2.92 | 30.64 | 9 |
| 2.89 | 30.89 | 40 |
| 2.78 | 32.22 | 3 |
| 2.76 | 32.41 | 2 |
| 2.73 | 32.73 | 5 |
| 2.62 | 34.20 | 10 |
| 2.57 | 34.81 | 16 |
| 2.56 | 34.96 | 7 |
| 2.51 | 35.76 | 4 |
| 2.49 | 36.07 | 6 |
| 2.46 | 36.46 | 3 |
| 2.38 | 37.82 | 2 |
| 2.35 | 38.31 | 4 |
| 2.30 | 39.16 | 9 |
| 2.28 | 39.51 | 7 |

### Example 2

### Formation of the Cinnamates

2a:

Batch: 800.5 mg (3.2 mmol) of (+)-(1R,2R)-3-[2-(dimethylamino)methyl-1-hydroxycyclohexyl]phenol; 476.6 mg (3.2 mmol) of cinnamic acid; 40 ml of dichloromethane. All solids were weighed into the reaction vessel. The solvent was added and the mixture was stirred at 30° C. for 17 hours by means of a vortexer (400 rpm). After cooling to room temperature, the solid obtained was suction-filtered through a filter frit. It was dried at 50° C. in vacuo (at most 5 mbar).

Yield: 1.208 g (99.5%) of a white, finely crystalline solid.

Melting point (DSC): 183.6° C. (onset); 184.5° C. (peak); 139.5 J/g.

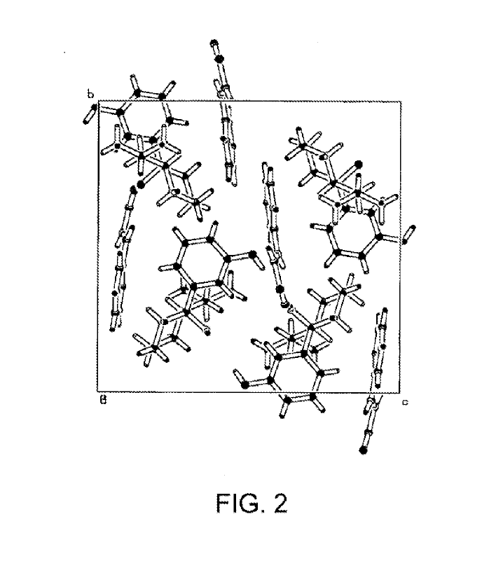

By slow cooling of an ethanolic solution of the cinnamate, monocrystals suitable for the X-ray structure determination were obtained. A structural element of the crystal structure is reproduced in FIG. 2. The substance is present, in accordance with expectation, as the cinnamate of the active ingredient. It crystallizes in the orthorhombic space group P2₁2₁2₁ widespread for enantiomerically pure substances. The asymmetric unit contains one cation and one anion each. Along the crystallographic a axis, cations and anions are linked by means of hydrogen bridges. FIG. 2 shows the arrangement of anions and cations within the crystal structure.

The following table reproduces the atomic coordinates (×10⁴) and the equivalent isotropic deflection parameters (Å²×10³). U(eq) is defined as one third of the track of the orthogonalized Uᵢⱼ tensor.

TABLE 14
Atomic coordinates

| | x | y | z | U(eq) |
|---|---|---|---|---|
| O(1) | 7733(1) | 2147(1) | 3598(1) | 18(1) |
| O(2) | 6076(1) | 4745(1) | 5115(1) | 28(1) |
| N(1) | 12043(1) | 3111(1) | 3585(1) | 16(1) |
| C(1) | 12613(1) | 3511(1) | 4375(1) | 20(1) |
| C(2) | 12744(1) | 3533(1) | 2845(1) | 23(1) |
| C(3) | 10385(1) | 3141(1) | 3578(1) | 18(1) |
| C(4) | 9653(1) | 2780(1) | 2784(1) | 18(1) |
| C(5) | 7954(1) | 2752(1) | 2934(1) | 16(1) |
| C(6) | 7176(1) | 2422(1) | 2132(1) | 22(1) |
| C(7) | 7715(1) | 1525(1) | 1878(1) | 24(1) |
| C(8) | 9385(1) | 1533(1) | 1753(1) | 26(1) |
| C(9) | 10200(1) | 1877(1) | 2528(1) | 22(1) |
| C(10) | 7379(1) | 3648(1) | 3192(1) | 18(1) |
| C(11) | 6945(1) | 3786(1) | 4025(1) | 18(1) |
| C(12) | 6465(1) | 4600(1) | 4298(1) | 23(1) |
| C(13) | 6379(1) | 5278(1) | 3724(1) | 35(1) |
| C(14) | 6811(2) | 5146(1) | 2901(1) | 40(1) |
| C(15) | 7318(1) | 4342(1) | 2627(1) | 30(1) |
| O(3) | 5179(1) | 6964(1) | 1186(1) | 30(1) |
| O(4) | 7442(1) | 6442(1) | 1015(1) | 21(1) |
| C(16) | 6042(1) | 6355(1) | 1014(1) | 19(1) |
| C(17) | 5477(1) | 5472(1) | 817(1) | 19(1) |
| C(18) | 4071(1) | 5242(1) | 934(1) | 17(1) |
| C(19) | 3471(1) | 4371(1) | 805(1) | 16(1) |
| C(20) | 4369(1) | 3633(1) | 792(1) | 19(1) |
| C(21) | 3765(1) | 2819(1) | 679(1) | 24(1) |
| C(22) | 2249(1) | 2722(1) | 570(1) | 26(1) |
| C(23) | 1341(1) | 3447(1) | 589(1) | 24(1) |
| C(24) | 1942(1) | 4265(1) | 711(1) | 20(1) |

2b:

Batch: 3.15 g (12.6 mmol) of (+)-(1R,2R)-3-[2-(dimethylamino)methyl-1-hydroxycyclohexyl]phenol; 1.9 g (12.6 mmol) of cinnamic acid; 63 ml of dichloromethane. All solids were weighed into the reaction vessel. The solvent was added and the mixture was stirred at 1000 rpm at room temperature for 25 hours. The solid obtained was filtered in a G4 glass funnel filter and sucked dry in air. It was dried in vacuo (at most 5 mbar) at 50° C.

Yield: 5.049 g (corresponds to 100.6% of the theoretical yield)

Melting point (DSC): 183.9° C. (onset); 184.6° C. (peak); 140.4 J/g.

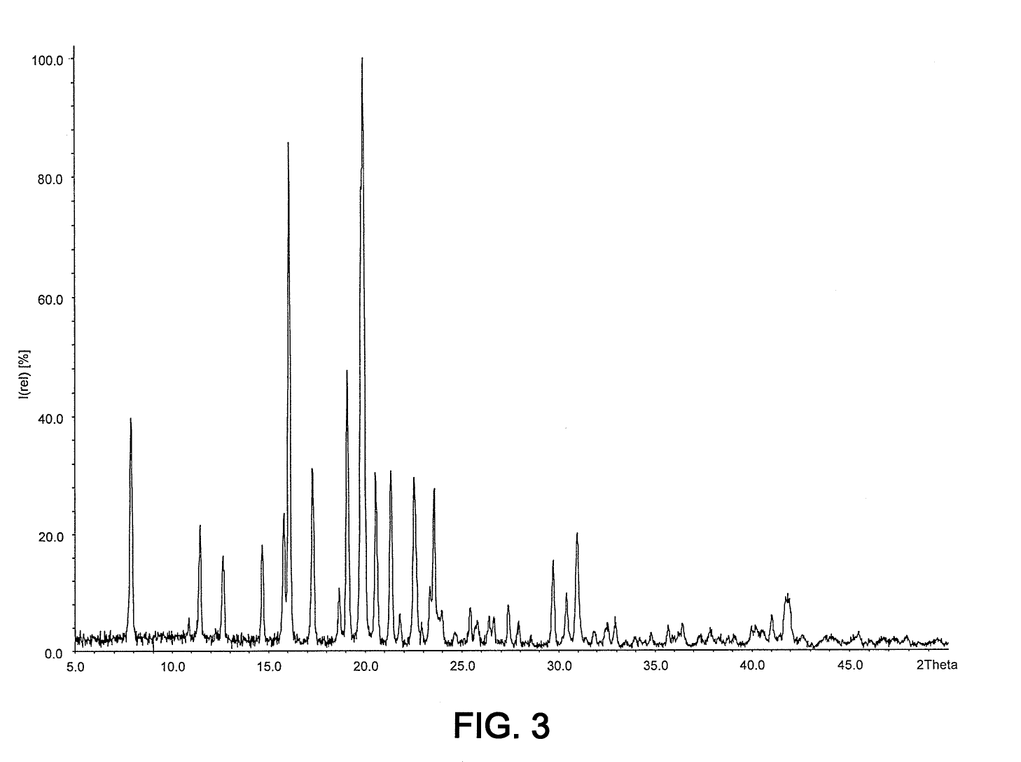

XRPD table of the product from batch 1a (shown graphically in FIG. 3):

TABLE 15
Powder diffractogram

| D | 2θ | I(rel) |
|---|---|---|
| 11.21 | 7.88 | 41 |
| 8.15 | 10.85 | 5 |
| 7.73 | 11.44 | 22 |
| 7.00 | 12.64 | 16 |
| 6.03 | 14.68 | 18 |
| 5.61 | 15.79 | 24 |
| 5.51 | 16.08 | 88 |
| 5.13 | 17.29 | 32 |
| 4.75 | 18.65 | 11 |
| 4.65 | 19.08 | 49 |
| 4.46 | 19.90 | 100 |
| 4.32 | 20.55 | 31 |
| 4.16 | 21.33 | 31 |
| 4.07 | 21.80 | 7 |
| 3.94 | 22.54 | 31 |
| 3.88 | 22.92 | 5 |
| 3.81 | 23.35 | 11 |
| 3.77 | 23.56 | 28 |
| 3.71 | 23.97 | 7 |
| 3.61 | 24.65 | 3 |
| 3.50 | 25.42 | 8 |
| 3.46 | 25.75 | 4 |
| 3.38 | 26.38 | 6 |
| 3.35 | 26.63 | 6 |
| 3.25 | 27.39 | 8 |
| 3.20 | 27.89 | 4 |
| 3.13 | 28.53 | 1 |
| 3.01 | 29.70 | 16 |
| 2.94 | 30.39 | 10 |
| 2.89 | 30.95 | 20 |
| 2.85 | 31.39 | 2 |
| 2.81 | 31.82 | 3 |
| 2.75 | 32.50 | 5 |
| 2.72 | 32.93 | 5 |
| 2.68 | 33.46 | 1 |
| 2.64 | 33.97 | 2 |
| 2.62 | 34.22 | 2 |
| 2.60 | 34.45 | 2 |
| 2.58 | 34.79 | 3 |
| 2.52 | 35.66 | 4 |
| 2.48 | 36.19 | 3 |
| 2.47 | 36.42 | 5 |
| 2.41 | 37.32 | 2 |
| 2.38 | 37.83 | 3 |
| 2.35 | 38.26 | 2 |
| 2.32 | 38.75 | 2 |
| 2.30 | 39.08 | 2 |
| 2.25 | 39.96 | 4 |

### 2c (Further Experiments on Formation of the Cinnamates):

In a multiple reactor, the reaction batches described below were carried out with (+)-(1R,2R)-3-[2-(dimethylamino)methyl-1-hydroxycyclohexyl]phenol and cinnamic acid using 1 ml of the respective solvent. The mixture was mixed for 4 hours at 30° C. and 700 rpm on a vortexer. Subsequently, the solvents were blown off at room temperature (22° C.) overnight in the stream of nitrogen of an evaporation station. A yield calculation was not carried out.

TABLE 16
Reaction batches for Example 2c

| Initial weight | Initial weight | | |
|---|---|---|---|
| of free | of cinnamic | | |
| base [mg] | acid [mg] | Solvent | Result |
| 5.01 | 3.09 | Acetone | white solid |
| 5.18 | 3.13 | dichloromethane | white solid |
| 5.17 | 3.11 | ethyl acetate | white solid |
| 5.15 | 3.18 | Isopropanol | white solid/needles |

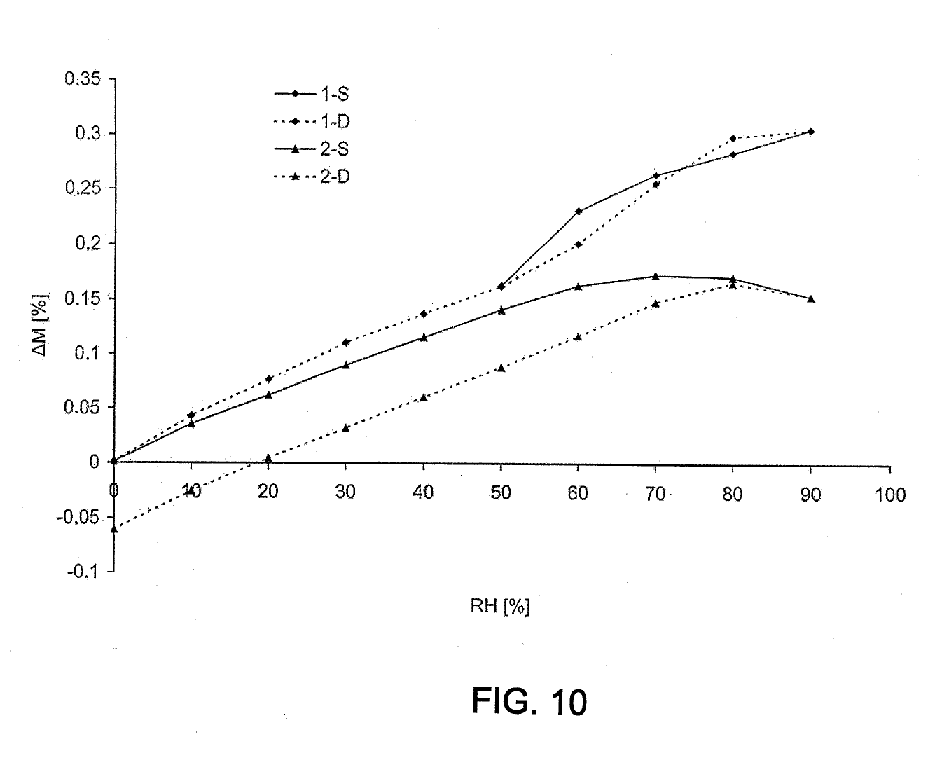

The dynamic vapor sorption spectrum of a cinnamate is reproduced in FIG. 10. The measurements are found in the following table. A third partial cycle not shown in the figure is additionally listed.

TABLE 17
DVS data

ΔM [%]

| | RH [%] | Sorption | Desorption | Hysteresis |
|---|---|---|---|---|
| 1st cycle | 0.0 | | 0.0012 | |
| | 10.0 | | 0.0432 | |
| | 20.0 | | 0.0766 | |
| | 30.0 | | 0.1106 | |
| | 40.0 | | 0.1370 | |
| | 50.0 | 0.1629 | 0.1624 | −0.0006 |
| | 60.0 | 0.2309 | 0.2010 | −0.0299 |
| | 70.0 | 0.2643 | 0.2562 | −0.0081 |
| | 80.0 | 0.2839 | 0.2988 | 0.0150 |
| | 90.0 | 0.3057 | 0.3057 | |
| 2nd cycle | 0.0 | 0.0012 | −0.0605 | |
| | 10.0 | 0.0357 | −0.0253 | −0.0610 |
| | 20.0 | 0.0622 | 0.0046 | −0.0576 |
| | 30.0 | 0.0898 | 0.0322 | −0.0576 |
| | 40.0 | 0.1157 | 0.0605 | −0.0553 |
| | 50.0 | 0.1411 | 0.0881 | −0.0530 |
| | 60.0 | 0.1629 | 0.1169 | −0.0461 |
| | 70.0 | 0.1727 | 0.1480 | −0.0248 |
| | 80.0 | 0.1710 | 0.1658 | −0.0052 |
| | 90.0 | 0.1532 | 0.1532 | |
| 3rd cycle | 0.0 | −0.0605 | | |
| | 10.0 | −0.0190 | | |
| | 20.0 | 0.0104 | | |
| | 30.0 | 0.0345 | | |
| | 40.0 | 0.0599 | | |
| | 50.0 | 0.0829 | | |

### Example 3

### Formation of the Palmitates

3a:

In a multiple reactor, the reaction batches described below were carried out with (+)-(1R,2R)-3-[2-(dimethylamino)methyl-1-hydroxycyclohexyl]phenol and palmitic acid using 1 ml of the respective solvent. The mixture was mixed on a vortexer for 4 hours at 30° C. and 700 rpm. Subsequently, the solvents were blown off at room temperature (22° C.) for 4 hours in the nitrogen flow of an evaporation station. After 64 hours, the results were appraised. A yield calculation was not carried out.

TABLE 18
Reaction batches for Example 3a

| Initial weight | Initial weight | | |
|---|---|---|---|
| of free | of palmitic | | |
| base [mg] | acid [mg] | Solvent | Result |
| 5.11 | 5.27 | acetone | white solid |
| 4.97 | 5.14 | dichloromethane | white solid |
| 5.09 | 5.25 | ethyl acetate | white solid |
| 4.93 | 4.99 | isopropanol | white solid |

3b:

Batch: 2.5 g (10 mmol) of (+)-(1R,2R)-3-[2-(dimethylamino)methyl-1-hydroxycyclohexyl]phenol; 2.6 g (10 mmol) of palmitic acid; 80 ml of n-pentane. All solids were weighed into the reaction vessel. The solvent was added and the mixture was stirred for 6 hours at 30° C. and 245 rpm. It was subsequently stirred at room temperature for 18.5 hours. The solid obtained was filtered through a G3 glass filter frit. It was dried at 50° C. in vacuo (5-7 mbar).

Yield: 4.97 g (corresponds to 98.0% of the theoretical yield)

Melting point (DSC): 78.7° C. (onset); 82.2° C. (peak); 97.0 J/g.

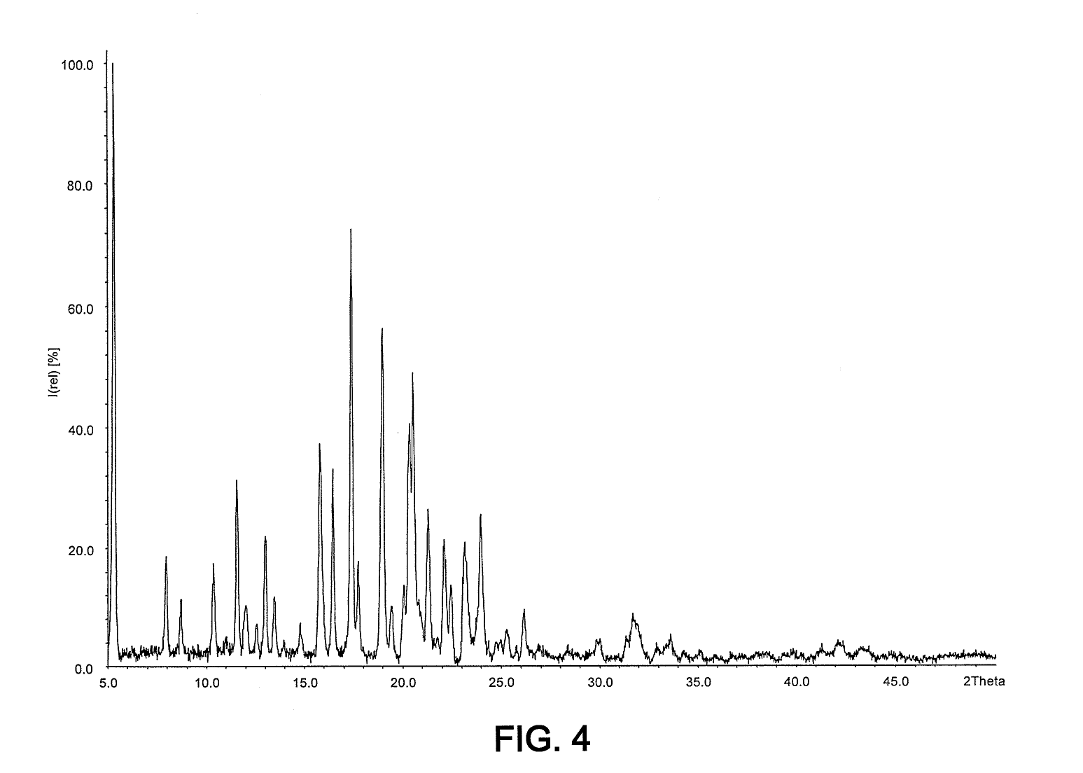

XRPD table of the product from batch 3b (shown graphically in FIG. 4):

TABLE 19
Powder diffractogram

| D | 2θ | I(rel) |
|---|---|---|
| 16.63 | 5.31 | 100 |
| 11.12 | 7.94 | 18 |
| 10.16 | 8.69 | 11 |
| 8.54 | 10.35 | 16 |
| 8.05 | 10.98 | 4 |
| 7.65 | 11.56 | 30 |
| 7.37 | 12.00 | 10 |
| 7.05 | 12.55 | 7 |
| 6.81 | 13.00 | 22 |
| 6.57 | 13.47 | 12 |
| 6.34 | 13.95 | 4 |
| 5.99 | 14.77 | 7 |
| 5.61 | 15.79 | 37 |
| 5.38 | 16.45 | 33 |
| 5.09 | 17.40 | 70 |
| 4.99 | 17.75 | 18 |
| 4.67 | 18.98 | 56 |
| 4.57 | 19.43 | 10 |
| 4.42 | 20.07 | 14 |
| 4.36 | 20.35 | 39 |
| 4.32 | 20.53 | 48 |
| 4.25 | 20.88 | 9 |
| 4.17 | 21.30 | 25 |
| 4.08 | 21.76 | 5 |
| 4.02 | 22.11 | 20 |
| 3.95 | 22.47 | 14 |
| 3.84 | 23.17 | 20 |
| 3.71 | 23.97 | 25 |
| 3.60 | 24.74 | 4 |
| 3.52 | 25.28 | 6 |
| 3.46 | 25.76 | 3 |
| 3.40 | 26.16 | 9 |
| 3.31 | 26.95 | 3 |
| 3.14 | 28.38 | 3 |
| 2.99 | 29.84 | 4 |
| 2.85 | 31.34 | 5 |
| 2.82 | 31.70 | 8 |
| 2.80 | 31.97 | 7 |
| 2.72 | 32.93 | 3 |
| 2.66 | 33.62 | 5 |
| 2.61 | 34.27 | 2 |
| 2.56 | 35.08 | 2 |

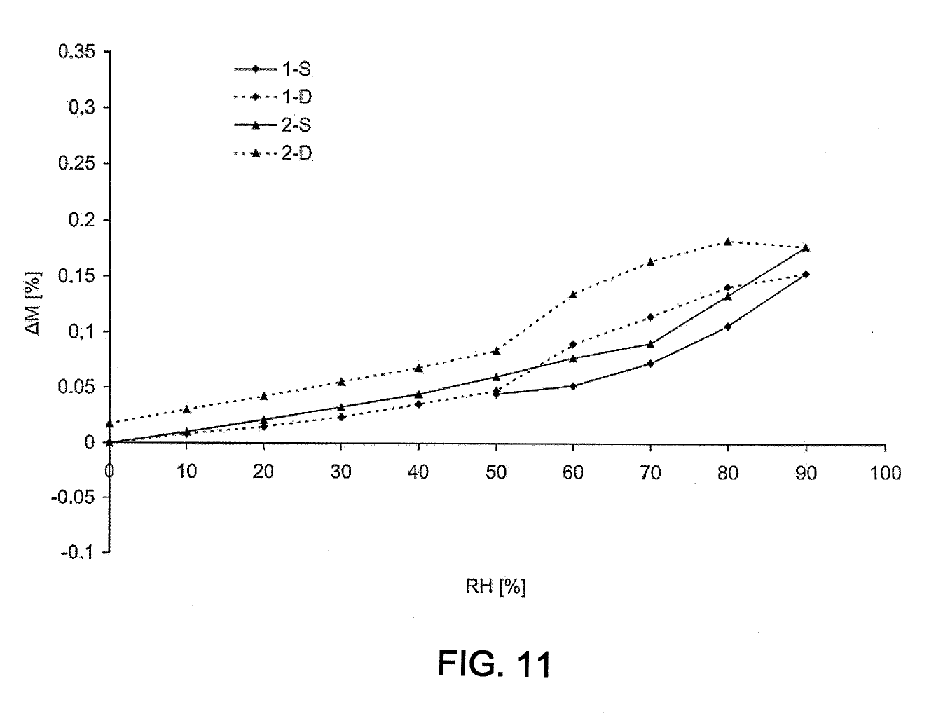

The dynamic vapor sorption spectrum of a palmitate is reproduced in FIG. 11. The measurements are found in the following table. A third partial cycle not shown in the figure is additionally presented.

TABLE 20
DVS data

ΔM [%]

| | RH [%] | Sorption | Desorption | Hysteresis |
|---|---|---|---|---|
| 1st cycle | 0.0 | | 0.0000 | |
| | 10.0 | | 0.0083 | |
| | 20.0 | | 0.0148 | |
| | 30.0 | | 0.0234 | |
| | 40.0 | | 0.0353 | |
| | 50.0 | 0.0445 | 0.0472 | 0.0027 |
| | 60.0 | 0.0522 | 0.0896 | 0.0374 |
| | 70.0 | 0.0727 | 0.1139 | 0.0412 |
| | 80.0 | 0.1059 | 0.1406 | 0.0347 |
| | 90.0 | 0.1531 | 0.1531 | |
| 2nd cycle | 0.0 | 0.0000 | 0.0172 | |
| | 10.0 | 0.0101 | 0.0303 | 0.0202 |
| | 20.0 | 0.0211 | 0.0421 | 0.0211 |
| | 30.0 | 0.0326 | 0.0552 | 0.0225 |
| | 40.0 | 0.0442 | 0.0679 | 0.0237 |
| | 50.0 | 0.0602 | 0.0837 | 0.0234 |
| | 60.0 | 0.0771 | 0.1344 | 0.0573 |
| | 70.0 | 0.0902 | 0.1640 | 0.0739 |
| | 80.0 | 0.1332 | 0.1827 | 0.0495 |
| | 90.0 | 0.1771 | 0.1771 | |
| 3rd cycle | 0.0 | 0.0172 | | |
| | 10.0 | 0.0231 | | |
| | 20.0 | 0.0306 | | |
| | 30.0 | 0.0392 | | |
| | 40.0 | 0.0498 | | |
| | 50.0 | 0.0650 | | |

### Example 4

### Formation of the Octanoates

4a:

In a multiple reactor, the reaction batches described below were carried out with (+)-(1R,2R)-3-[2-(dimethylamino)methyl-1-hydroxycyclohexyl]phenol and caprylic acid (octanoic acid) using 1 ml of the respective solvent. The mixture was mixed on a vortexer for 4 hours at 30° C. and 700 rpm. Subsequently, the solvents were blown off at room temperature (22° C.) for 4 hours in the nitrogen flow of an evaporation station. After 64 hours, the results were appraised. A yield calculation was not carried out.

TABLE 21
Reaction batches for Example 4a

| Initial weight | Initial weight | | |
|---|---|---|---|
| of free | of octanoic | | |
| base [mg] | acid [mg] | Solvent | Result |
| 4.99 | 3.18 | acetone | white solid and |
| | | | colorless oil |
| 4.97 | 3.18 | dichloromethane | white solid |
| 4.92 | 3.18 | ethyl acetate | colorless oil and |
| | | | white solid |
| 5.07 | 3.18 | isopropanol | colorless oil and |
| | | | crystal seeds |

4b:

Batch: 3.3 g (13.2 mmol) of (+)-(1R,2R)-3-[2-(dimethylamino)methyl-1-hydroxycyclohexyl]phenol were initially introduced in 70 ml of tert-butyl methyl ether and treated with 1.9 g (13.2 mmol) of octanoic acid. A clouding of the reaction mixture was observed. The suspension obtained was mixed on a vortexer at 23° C. and 800 rpm for 22 hours. The solid obtained was filtered through a G3 glass filter frit. It was dried at 50° C. in vacuo (5 mbar).

Yield: 4.01 g (corresponds to 78.5% of the theoretical yield)

Melting point (DSC): 100.5° C. (onset); 102.2° C. (peak); 123.7 J/g.

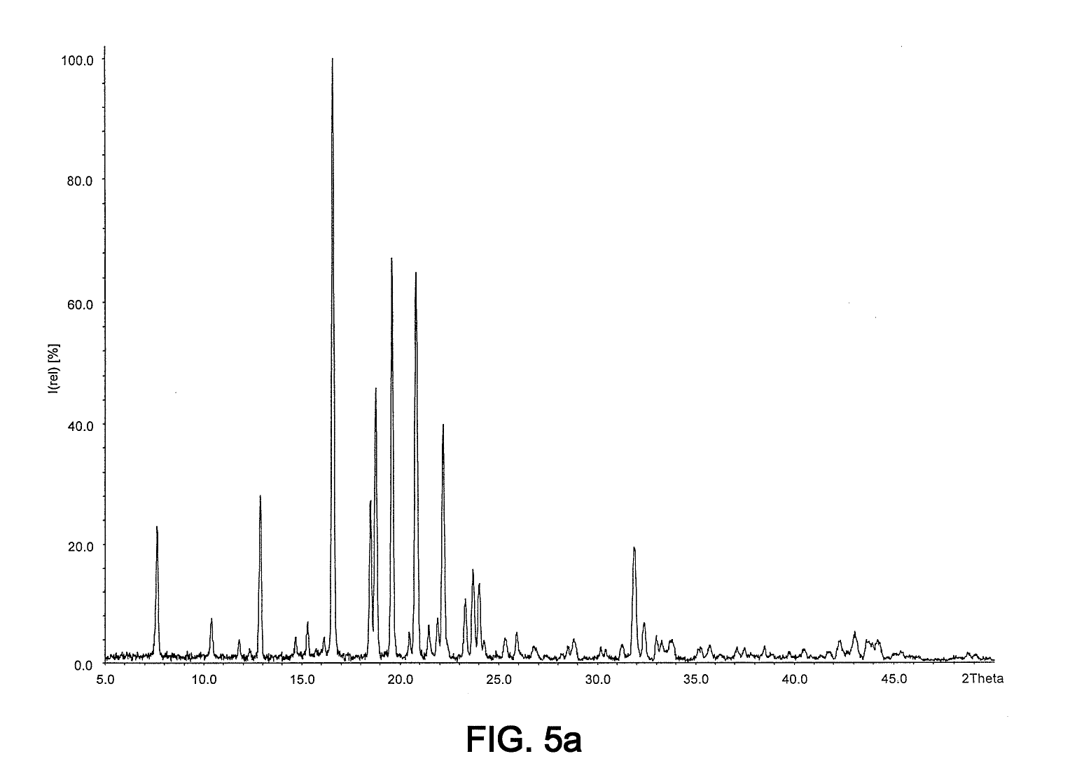

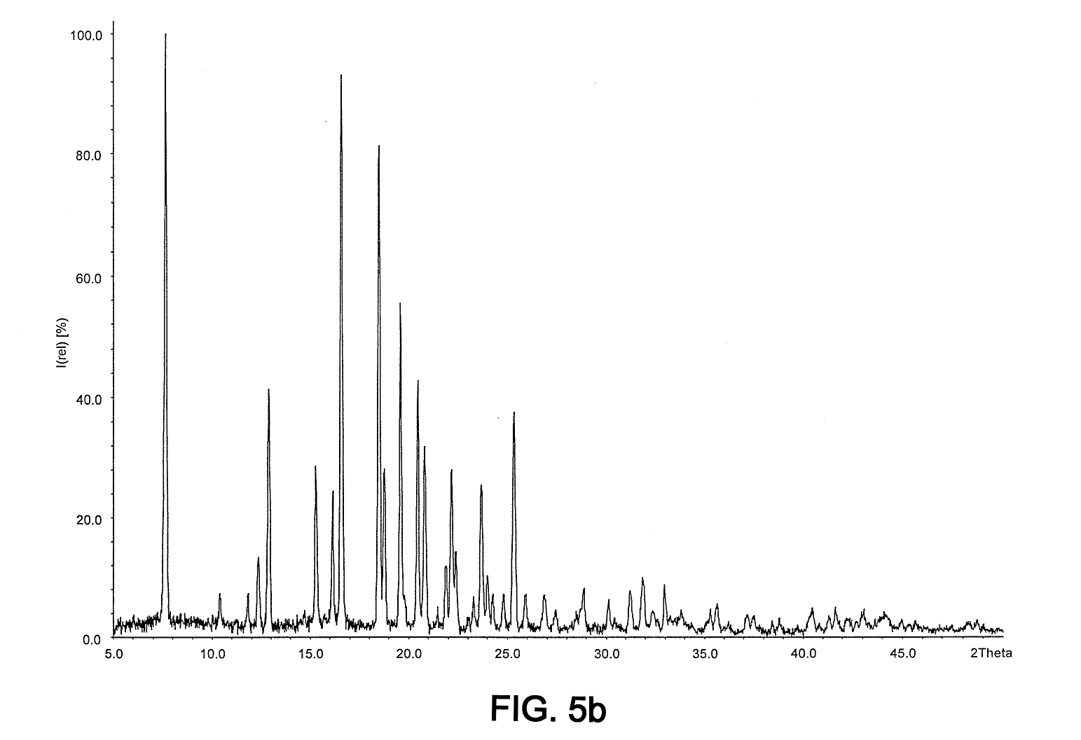

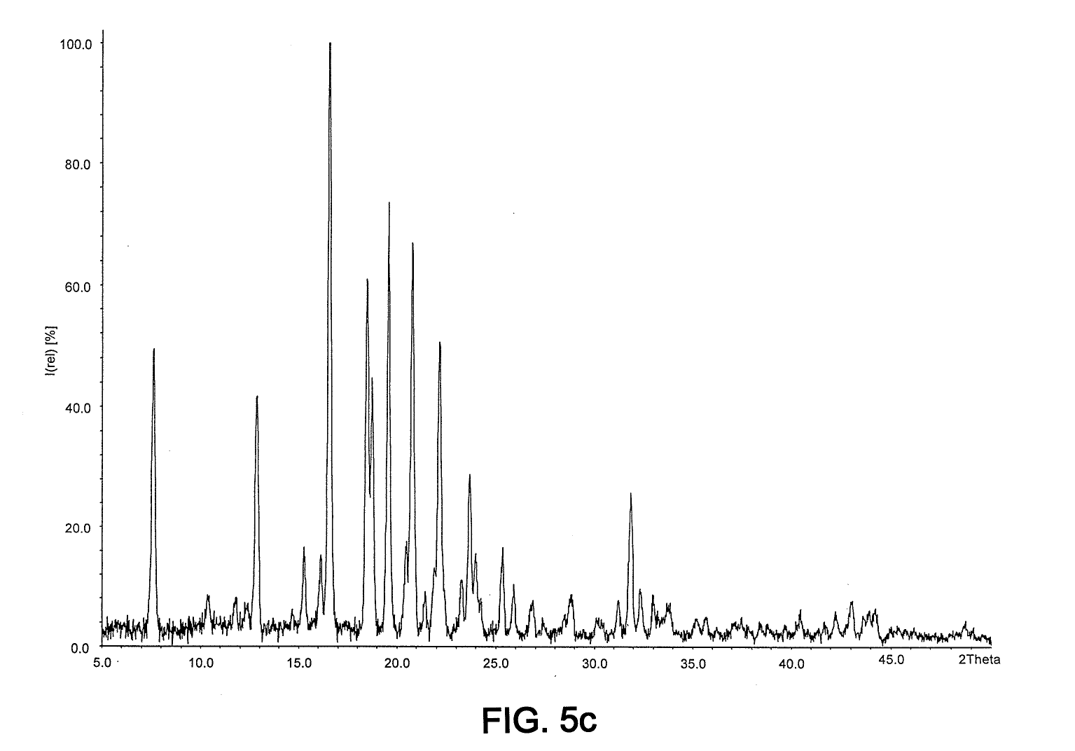

XRPD table of the product from batch 4b (reproduced graphically in FIG. 5a):

TABLE 22
Powder diffractogram

| D | 2θ | I(rel) |
|---|---|---|
| 11.58 | 7.63 | 23 |
| 8.51 | 10.38 | 7 |
| 7.50 | 11.79 | 4 |
| 7.18 | 12.33 | 2 |
| 6.87 | 12.87 | 28 |
| 6.03 | 14.68 | 4 |
| 5.79 | 15.30 | 7 |
| 5.64 | 15.71 | 2 |
| 5.49 | 16.13 | 4 |
| 5.34 | 16.59 | 100 |
| 4.79 | 18.50 | 27 |
| 4.72 | 18.77 | 45 |
| 4.53 | 19.60 | 68 |
| 4.34 | 20.46 | 5 |
| 4.26 | 20.81 | 65 |
| 4.14 | 21.45 | 6 |
| 4.06 | 21.89 | 7 |
| 4.00 | 22.18 | 40 |
| 3.81 | 23.30 | 10 |
| 3.75 | 23.70 | 15 |
| 3.70 | 24.01 | 13 |
| 3.67 | 24.26 | 4 |
| 3.58 | 24.85 | 2 |
| 3.52 | 25.32 | 4 |
| 3.44 | 25.91 | 5 |
| 3.33 | 26.78 | 3 |
| 3.25 | 27.41 | 1 |
| 3.16 | 28.20 | 1 |
| 3.13 | 28.51 | 3 |
| 3.10 | 28.80 | 4 |
| 2.96 | 30.18 | 3 |
| 2.94 | 30.41 | 2 |
| 2.86 | 31.25 | 3 |
| 2.80 | 31.88 | 20 |
| 2.76 | 32.38 | 7 |
| 2.71 | 33.00 | 4 |
| 2.69 | 33.26 | 4 |
| 2.65 | 33.77 | 4 |
| 2.55 | 35.23 | 2 |
| 2.51 | 35.72 | 3 |
| 2.42 | 37.10 | 3 |
| 2.40 | 37.48 | 2 |
| 2.34 | 38.50 | 3 |

The mother liquor of the filtrate from this example was completely freed of solvent. 1.0321 g (19.8% of the theoretical yield) were obtained. The solid obtained was likewise characterized.

Melting point (DSC): 100.5° C. (onset); 102.5° C. (peak); 113.26 J/g.

The X-ray powder diffractogram spectrum of this product is reproduced graphically in FIG. 5b.

A further X-ray powder diffractogram spectrum of the octanoate is reproduced graphically in FIG. 5c.

### Example 5

### Formation of the Decanoates

5a:

In a multiple reactor, the reaction batches described below were carried out with (+)-(1R,2R)-3-[2-(dimethylamino)methyl-1-hydroxycyclohexyl]phenol and capric acid (decanoic acid) using 1 ml of the respective solvent. The mixture was mixed on a vortexer for 4 hours at 30° C. and 700 rpm. Subsequently, the solvents were blown off at room temperature (22° C.) for 4 hours in the nitrogen flow of an evaporation station. After 64 hours, the results were appraised. A yield calculation was not carried out.

TABLE 23
Reaction batches for Example 5a

| Initial weight | Initial weight | | |
|---|---|---|---|
| of free | of decanoic | | |
| base [mg] | acid [mg] | Solvent | Result |
| 5.1 | 3.46 | acetone | colorless oil and |
| | | | crystal seeds |
| 4.99 | 3.44 | dichloromethane | white solid; white, |
| | | | lustrous crystal |
| | | | faces |
| 4.95 | 3.45 | ethyl acetate | white solid; white, |
| | | | lustrous crystal |
| | | | faces |
| 5.07 | 3.5 | isopropanol | white solid; white, |
| | | | lustrous crystal |
| | | | faces |

5b:

Batch: 3.0 g (12 mmol) of (+)-(1R,2R)-3-[2-(dimethylamino)methyl-1-hydroxycyclohexyl]phenol; 2.1 g (12 mmol) of decanoic acid; 80 ml of n-pentane. All solids were weighed into the reaction vessel. The solvent was added and the mixture was stirred for 6 hours at 30° C. and 245 rpm. It was then stirred at room temperature for 18.5 hours. The solid obtained was filtered through a G3 glass filter frit. It was dried at 50° C. in vacuo (5-7 mbar).

Yield: 5.0 g (corresponds to 99.2% of the theoretical yield)

Melting point (DSC): 95.5° C. (onset); 97.6° C. (peak); 89.0 J/g.

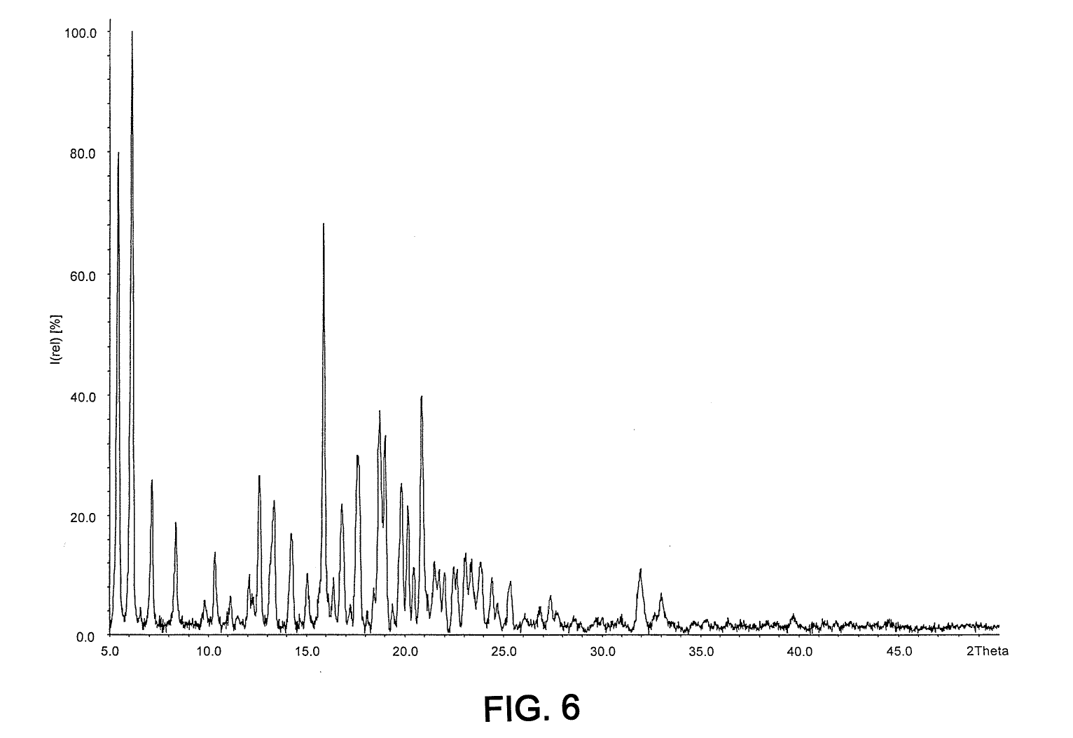

XRPD table of the product from batch 5b (reproduced graphically in FIG. 6):

TABLE 24
Powder diffractogram

| D | 2θ | I(rel) |
|---|---|---|
| 16.30 | 5.42 | 80 |
| 14.44 | 6.12 | 100 |
| 13.47 | 6.56 | 4 |
| 12.39 | 7.13 | 26 |
| 10.59 | 8.34 | 19 |
| 9.00 | 9.82 | 6 |
| 8.55 | 10.34 | 14 |
| 7.95 | 11.12 | 6 |
| 7.70 | 11.49 | 3 |
| 7.33 | 12.07 | 9 |
| 7.22 | 12.25 | 7 |
| 7.02 | 12.60 | 27 |
| 6.64 | 13.32 | 22 |
| 6.23 | 14.21 | 17 |
| 5.89 | 15.03 | 10 |
| 5.58 | 15.88 | 68 |
| 5.41 | 16.36 | 9 |
| 5.27 | 16.80 | 22 |
| 5.14 | 17.23 | 4 |
| 5.03 | 17.63 | 30 |
| 4.90 | 18.09 | 4 |
| 4.81 | 18.44 | 7 |
| 4.73 | 18.73 | 36 |
| 4.66 | 19.01 | 33 |
| 4.58 | 19.38 | 5 |
| 4.47 | 19.83 | 26 |
| 4.40 | 20.15 | 22 |
| 4.34 | 20.44 | 11 |
| 4.25 | 20.86 | 40 |
| 4.19 | 21.18 | 7 |
| 4.13 | 21.50 | 12 |
| 4.08 | 21.75 | 11 |
| 4.04 | 22.00 | 10 |
| 3.95 | 22.48 | 11 |
| 3.93 | 22.62 | 10 |
| 3.85 | 23.06 | 13 |
| 3.81 | 23.35 | 11 |
| 3.73 | 23.83 | 12 |
| 3.64 | 24.40 | 9 |
| 3.60 | 24.68 | 5 |
| 3.51 | 25.33 | 9 |
| 3.42 | 26.06 | 3 |
| 3.32 | 26.81 | 4 |
| 3.25 | 27.38 | 6 |
| 3.22 | 27.70 | 3 |
| 3.12 | 28.55 | 3 |
| 3.01 | 29.70 | 3 |
| 2.98 | 30.00 | 3 |
| 2.89 | 30.86 | 2 |
| 2.80 | 31.94 | 11 |
| 2.74 | 32.69 | 3 |
| 2.71 | 33.03 | 6 |
| 2.59 | 34.67 | 2 |
| 2.54 | 35.27 | 3 |
| 2.46 | 36.43 | 2 |
| 2.27 | 39.71 | 3 |

### Example 6

### Formation of the Hemi-Fumarates

6a:

In a multiple reactor, the reaction batches described below were carried out with (+)-(1R,2R)-3-[2-(dimethylamino)methyl-1-hydroxycyclohexyl]phenol and fumaric acid using 1 ml of the respective solvent. The mixture was mixed on a vortexer for 4 hours at 30° C. and 700 rpm. Subsequently, the solvents were blown off at room temperature (22° C.) overnight in the nitrogen flow of an evaporation station. A yield calculation was not carried out.

TABLE 25
Reaction batches for Example 6a

| Initial weight | Initial weight | | |
|---|---|---|---|
| of free | of fumaric | | |
| base [mg] | acid [mg] | Solvent | Result |
| 5.17 | 2.44 | acetone | clear oil |
| 5.01 | 2.49 | dichloromethane | white solid |
| 5.12 | 2.6 | ethyl acetate | white crystals |
| 5.09 | 2.43 | isopropanol | white solid |

6b:

Batch: 4.1 g (16.4 mmol) of (+)-(1R,2R)-3-[2-(dimethylamino)methyl-1-hydroxycyclohexyl]phenol; 0.954 g (8.2 mmol) of fumaric acid; 80 ml of isopropanol. All solids were weighed into the reaction vessel. The solvent was added and the mixture was stirred for 23 hours at 23° C. and 800 rpm. The solid obtained was filtered through a G3 glass filter frit. It was dried to constant mass at 50° C. in vacuo (5 mbar).

Yield: 5.99 g (corresponds to 118% of the theoretical yield; a thermogravimetric investigation of the sample showed a mass loss of about 19% in the range from 90 to 180° C., which suggests the presence of solvent)

Melting point (DSC): 1) 62.8° C. (onset); 89.3° C. (peak); 119.2 J/g
2) 93.3° C. (onset); 97.6° C. (peak); 68.6 J/g
3) 130.1° C. (onset); 137.8° C. (peak); 15.2 J/g

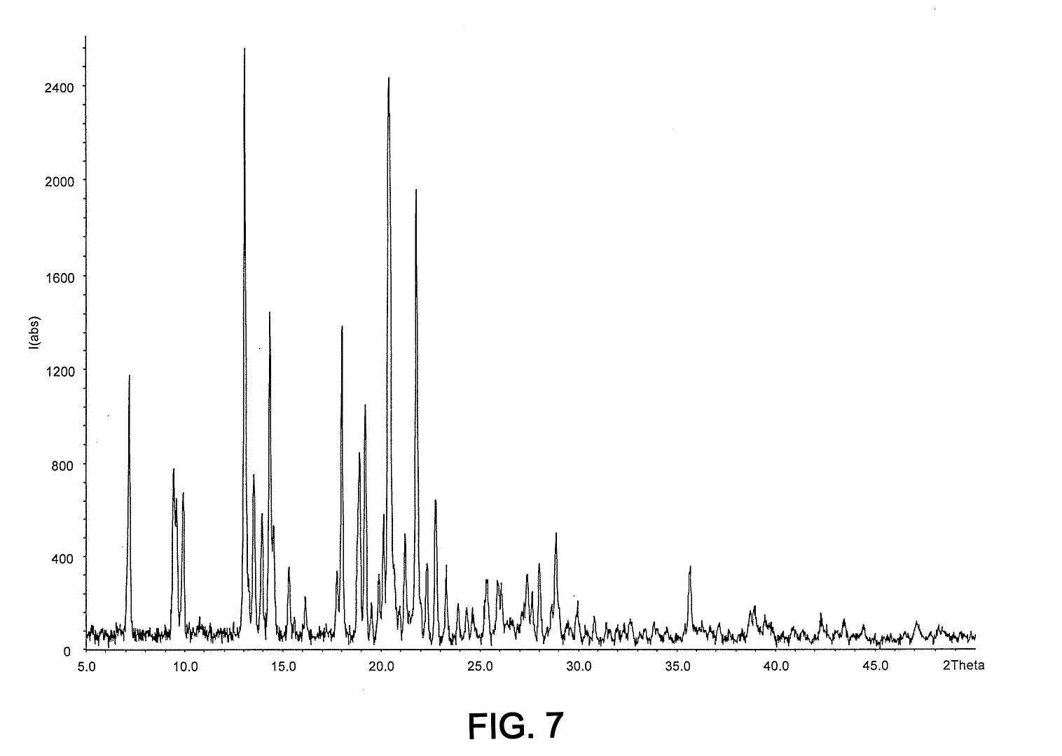

XRPD table of the product from batch 6b (reproduced graphically in FIG. 7):

TABLE 26
Powder diffractogram

| D | 2θ | I(rel) |
|---|---|---|
| 12.33 | 7.16 | 46 |
| 9.37 | 9.43 | 31 |
| 9.27 | 9.54 | 24 |
| 8.92 | 9.91 | 26 |
| 6.77 | 13.06 | 100 |
| 6.55 | 13.51 | 29 |
| 6.36 | 13.92 | 23 |
| 6.18 | 14.33 | 56 |
| 6.11 | 14.50 | 21 |
| 5.79 | 15.30 | 14 |
| 5.69 | 15.57 | 5 |
| 5.49 | 16.13 | 9 |
| 4.99 | 17.76 | 13 |
| 4.92 | 18.01 | 53 |
| 4.69 | 18.89 | 32 |
| 4.62 | 19.19 | 42 |
| 4.55 | 19.50 | 8 |
| 4.46 | 19.88 | 12 |
| 4.41 | 20.12 | 20 |
| 4.35 | 20.42 | 96 |
| 4.30 | 20.65 | 14 |
| 4.24 | 20.92 | 6 |
| 4.18 | 21.22 | 20 |
| 4.15 | 21.40 | 6 |
| 4.07 | 21.80 | 75 |
| 3.98 | 22.31 | 14 |
| 3.90 | 22.76 | 26 |
| 3.82 | 23.29 | 13 |
| 3.72 | 23.89 | 8 |
| 3.66 | 24.33 | 6 |
| 3.61 | 24.62 | 6 |
| 3.51 | 25.34 | 11 |
| 3.44 | 25.90 | 12 |
| 3.41 | 26.08 | 11 |
| 3.37 | 26.45 | 4 |
| 3.28 | 27.13 | 6 |
| 3.25 | 27.39 | 13 |
| 3.22 | 27.64 | 9 |
| 3.18 | 28.01 | 15 |
| 3.12 | 28.63 | 7 |
| 3.09 | 28.84 | 18 |
| 3.03 | 29.42 | 4 |
| 2.98 | 29.95 | 7 |
| 2.94 | 30.34 | 3 |
| 2.90 | 30.81 | 5 |
| 2.85 | 31.40 | 5 |
| 2.83 | 31.57 | 3 |
| 2.80 | 31.97 | 4 |
| 2.77 | 32.30 | 3 |
| 2.74 | 32.62 | 5 |
| 2.68 | 33.35 | 3 |
| 2.65 | 33.83 | 5 |
| 2.63 | 34.01 | 3 |
| 2.60 | 34.46 | 4 |
| 2.53 | 35.40 | 4 |
| 2.52 | 35.66 | 13 |
| 2.47 | 36.27 | 4 |
| 2.45 | 36.69 | 4 |
| 2.42 | 37.13 | 4 |
| 2.39 | 37.67 | 3 |
| 2.32 | 38.72 | 6 |
| 2.31 | 38.94 | 7 |
| 2.27 | 39.60 | 3 |

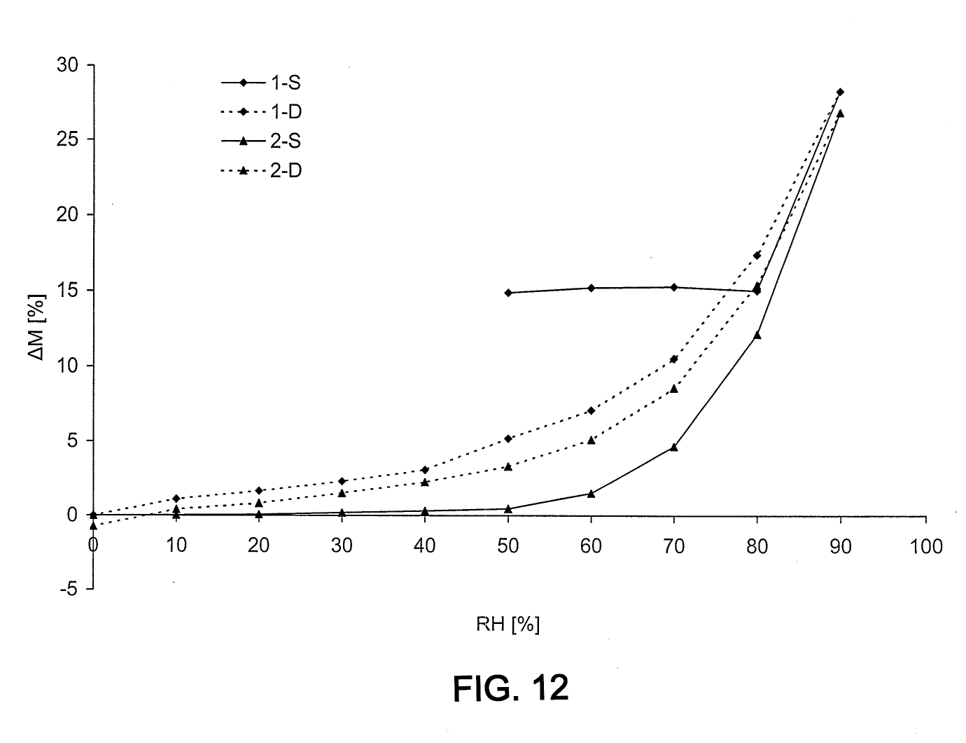

The dynamic vapor sorption spectrum of a hemi-fumarate is reproduced in FIG. 12. The measurements are found in the following table. A third partial cycle not shown in the figure is additionally presented.

TABLE 27
DVS data

ΔM [%]

| | RH [%] | Sorption | Desorption | Hysteresis |
|---|---|---|---|---|
| 1st cycle | 0.0 | | 0.00 | |
| | 10.0 | | 1.06 | |
| | 20.0 | | 1.62 | |
| | 30.0 | | 2.28 | |
| | 40.0 | | 3.06 | |
| | 50.0 | 14.87 | 5.19 | −9.67 |
| | 60.0 | 15.20 | 7.05 | −8.14 |
| | 70.0 | 15.27 | 10.49 | −4.77 |
| | 80.0 | 15.00 | 17.44 | 2.43 |
| | 90.0 | 28.28 | 28.28 | |
| 2nd cycle | 0.0 | 0.00 | −0.74 | |
| | 10.0 | 0.02 | 0.41 | 0.39 |
| | 20.0 | 0.08 | 0.81 | 0.73 |
| | 30.0 | 0.18 | 1.46 | 1.28 |
| | 40.0 | 0.30 | 2.24 | 1.94 |
| | 50.0 | 0.45 | 3.31 | 2.86 |
| | 60.0 | 1.48 | 5.10 | 3.62 |
| | 70.0 | 4.65 | 8.57 | 3.92 |
| | 80.0 | 12.11 | 15.39 | 3.27 |
| | 90.0 | 26.85 | 26.85 | |
| 3rd cycle | 0.0 | −0.74 | | |
| | 10.0 | −0.72 | | |
| | 20.0 | −0.67 | | |
| | 30.0 | −0.57 | | |
| | 40.0 | −0.46 | | |
| | 50.0 | −0.22 | | |

### Example 7

### Formation of the Hemi-Sebacates

7a:

In a multiple reactor, the reaction batches described below were carried out with (+)-(1R,2R)-3-[2-(dimethylamino)methyl-1-hydroxycyclohexyl]phenol and sebacic acid using 1 ml of the respective solvent. The mixture was mixed on a vortexer for 4 hours at 30° C. and 700 rpm. Subsequently, the solvents were blown off at room temperature (22° C.) overnight in the nitrogen flow of an evaporation station. A yield calculation was not carried out.

TABLE 28
Reaction batches for Example 7a

| Initial weight | Initial weight | | |
|---|---|---|---|
| of free | of sebacic | | |
| base [mg] | acid [mg] | Solvent | Result |
| 5.14 | 4.26 | acetone | white solid |
| 5.14 | 4.28 | dichloromethane | oil |
| 5.07 | 4.3 | ethyl acetate | oil |
| 5.08 | 4.19 | isopropanol | white solid |

7b:

Batch: 1.0 g (4 mmol) of (+)-(1R,2R)-3-[2-(dimethylamino)methyl-1-hydroxycyclohexyl]phenol; 0.405 g (2 mmol) of sebacic acid; 40 ml of tert-butyl methyl ether. All solids were weighed into the reaction vessel. The solvent was added and the mixture was mixed on a vortexer for 17 hours at 30° C. and 400 rpm. After cooling to room temperature, the solid obtained was filtered in a G4 glass filter funnel.

Yield: 1.37 g (corresponds to 98% of the theoretical yield)

Melting point (DSC): 159.3° C. (onset); 160.7° C. (peak); 162.6 J/g

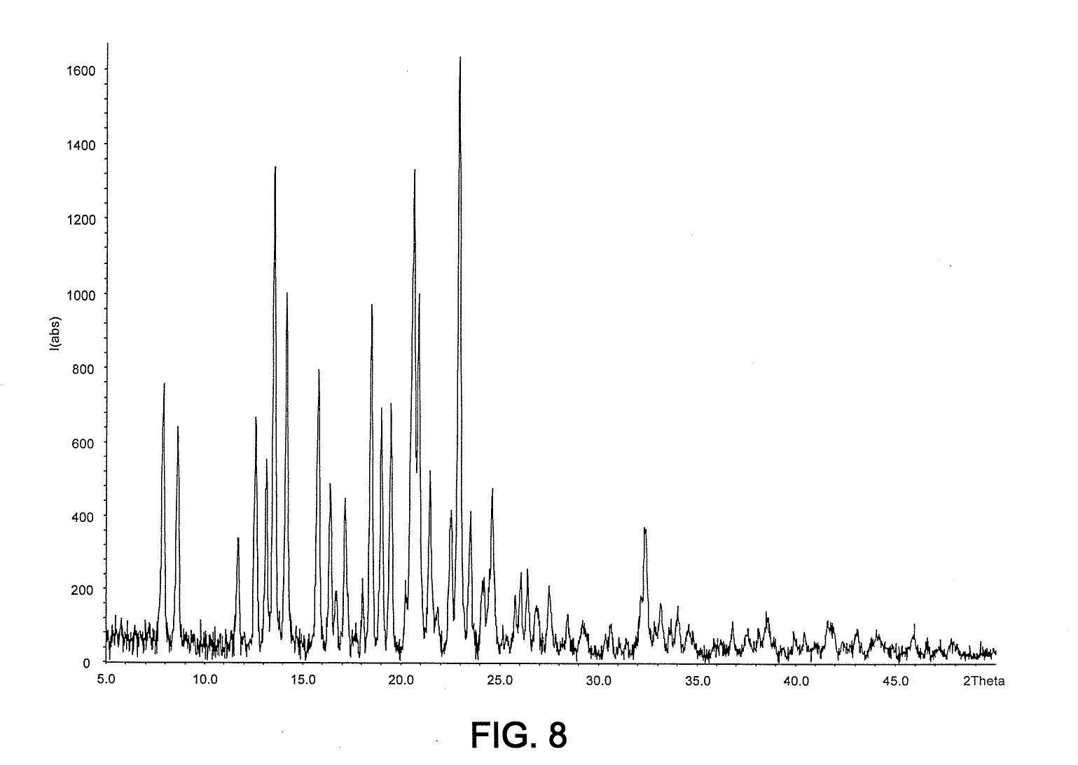

XRPD table of the product from batch 7b (reproduced graphically in FIG. 8):

TABLE 29
Powder diffractogram

| D | 2θ | I(rel) |
|---|---|---|
| 11.22 | 7.87 | 45 |
| 10.26 | 8.61 | 40 |
| 7.56 | 11.70 | 22 |
| 7.03 | 12.59 | 41 |
| 6.74 | 13.13 | 33 |
| 6.54 | 13.53 | 82 |
| 6.25 | 14.15 | 62 |
| 5.61 | 15.77 | 48 |
| 5.40 | 16.39 | 30 |
| 5.30 | 16.71 | 12 |
| 5.17 | 17.15 | 27 |
| 5.01 | 17.68 | 4 |
| 4.91 | 18.04 | 13 |
| 4.80 | 18.47 | 60 |
| 4.67 | 19.00 | 41 |
| 4.56 | 19.47 | 44 |
| 4.39 | 20.23 | 14 |
| 4.30 | 20.63 | 81 |
| 4.14 | 21.47 | 32 |
| 4.06 | 21.87 | 10 |
| 3.95 | 22.52 | 25 |
| 3.87 | 22.94 | 100 |
| 3.78 | 23.50 | 24 |
| 3.68 | 24.18 | 14 |
| 3.61 | 24.61 | 28 |
| 3.54 | 25.14 | 6 |
| 3.51 | 25.36 | 4 |
| 3.45 | 25.78 | 12 |
| 3.42 | 26.05 | 15 |
| 3.37 | 26.40 | 16 |
| 3.32 | 26.87 | 9 |
| 3.24 | 27.49 | 12 |
| 3.14 | 28.43 | 8 |
| 3.06 | 29.20 | 7 |
| 2.92 | 30.62 | 7 |
| 2.78 | 32.16 | 12 |
| 2.76 | 32.37 | 23 |
| 2.70 | 33.15 | 10 |
| 2.66 | 33.63 | 5 |
| 2.63 | 34.02 | 9 |
| 2.59 | 34.58 | 6 |
| 2.49 | 36.06 | 4 |
| 2.44 | 36.82 | 7 |
| 2.39 | 37.59 | 6 |
| 2.36 | 38.13 | 4 |
| 2.33 | 38.60 | 7 |
| 2.31 | 38.90 | 5 |
| 2.29 | 39.33 | 3 |
| 2.26 | 39.93 | 5 |

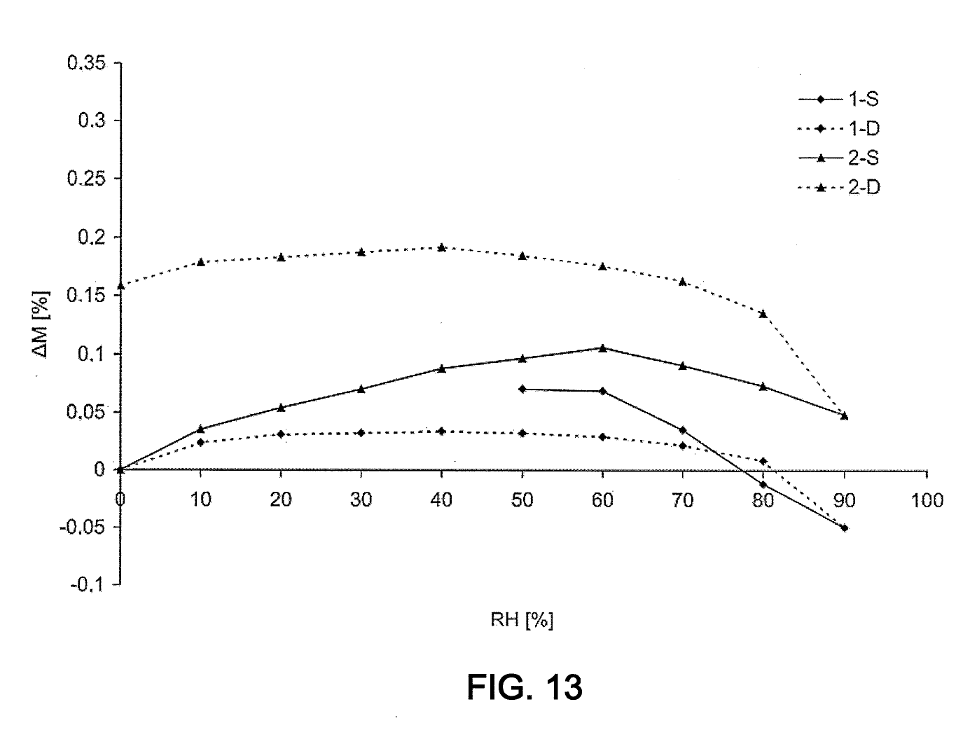

The dynamic vapor sorption spectrum of a hemi-sebacate is reproduced in FIG. 13. The measurements are found in the following table. A third partial cycle not shown in the figure is additionally presented.

TABLE 30
DVS data

ΔM [%]

| | RH [%] | Sorption | Desorption | Hysteresis |
|---|---|---|---|---|
| 1st cycle | 0.0 | | 0.0000 | |
| | 10.0 | | 0.0235 | |
| | 20.0 | | 0.0308 | |
| | 30.0 | | 0.0323 | |
| | 40.0 | | 0.0338 | |
| | 50.0 | 0.0704 | 0.0323 | −0.0382 |
| | 60.0 | 0.0690 | 0.0293 | −0.0396 |
| | 70.0 | 0.0352 | 0.0220 | −0.0132 |
| | 80.0 | −0.0117 | 0.0088 | 0.0205 |
| | 90.0 | −0.0499 | −0.0499 | |
| 2nd cycle | 0.0 | 0.0000 | 0.1585 | |
| | 10.0 | 0.0352 | 0.1790 | 0.1438 |
| | 20.0 | 0.0543 | 0.1834 | 0.1291 |
| | 30.0 | 0.0704 | 0.1878 | 0.1174 |
| | 40.0 | 0.0880 | 0.1922 | 0.1042 |
| | 50.0 | 0.0968 | 0.1849 | 0.0880 |
| | 60.0 | 0.1057 | 0.1761 | 0.0704 |
| | 70.0 | 0.0910 | 0.1629 | 0.0719 |
| | 80.0 | 0.0734 | 0.1350 | 0.0616 |
| | 90.0 | 0.0484 | 0.0484 | |
| 3rd cycle | 0.0 | 0.1585 | | |
| | 10.0 | 0.1937 | | |
| | 20.0 | 0.2113 | | |
| | 30.0 | 0.2245 | | |
| | 40.0 | 0.2377 | | |
| | 50.0 | 0.2465 | | |

### Example 8

### Determination of Equilibrium Solubilities

The equilibrium solubilities of the compounds investigated were determined in aqueous solutions at various pHs and the pH-dependent solubility profile and the solubilities at different pHs were compared here. In this case, the content determination was carried out by means of capillary electrophoresis.

The equilibrium solubilities, expressed in mmol/ml of the (+)-(1R,2R)-configured free base, are indicated below. In this case, the compounds denote: HCl: hydrochloride (comparison); A: cinnamate; B: palmitate; C: decanoate; D: octanoate; E: hemi-fumarate and F: hemi-sebacate. “nd”: not determined.

TABLE 31
Equilibrium solubilities

| | HCl | A | B | C | D | E | F |
|---|---|---|---|---|---|---|---|
| Water | 983 | 22 | 4 | 16 | 38 | >1627 | 575 |
| Water/ethanol | 1089 | 103 | 278 | >1186 | 688 | >1627 | 136 |
| 60/40 | | | | | | | |
| Buffer pH 1.4 | 582 | 17 | 12 | 90 | 95 | >1627 | 23 |
| Buffer pH 4.3 | 666 | 31 | 13 | 81 | 70 | >1627 | 75 |
| Buffer pH 6.8 | 698 | 19 | 23 | 54 | 32 | >1627 | 58 |
| Buffer pH 7.4 | 867 | 19 | 12 | 39 | 32 | >1627 | 56 |
| Buffer pH 11 | 979 | 15 | nd | 76 | 30 | >1627 | 58 |

### Example 9

### Release in Artificial Intestinal Juice

In this release experiment, the individual compounds were placed in a permeable bag in an amount in each case calculated on 100 mg of the free base.

The results, expressed in percent of the substance released, are reproduced in the following tables. In this case, the compounds denote: HCl: hydrochloride; A: cinnamate; B: palmitate; C: decanoate; E: hemi-fumarate and F: hemi-sebacate.

TABLE 32
Release in artificial intestinal juice

| t [min] | HCl-1 | HCl-2 | A-1 | A-2 | B-1 | B-2 |
|---|---|---|---|---|---|---|
| 2 | 99.16 | 97.72 | 58.49 | 53.05 | 17.63 | 37.44 |
| 4 | 99.89 | 98.52 | 74.11 | 76.86 | 31.60 | 187.50 |
| 6 | 98.77 | 100.41 | 84.48 | 86.98 | 55.36 | 68.02 |
| 8 | 101.08 | 98.83 | 91.49 | 96.27 | 53.71 | 55.66 |
| 10 | 100.57 | 100.29 | 92.24 | 94.24 | 67.26 | 71.79 |
| 12 | 100.66 | 98.70 | 96.63 | 95.42 | 69.49 | 78.62 |
| 15 | 99.62 | 100.05 | 97.34 | 98.53 | 76.13 | 83.30 |
| 30 | 101.02 | 99.96 | 102.44 | 98.80 | 95.50 | 131.23 |

TABLE 33
Release in artificial intestinal juice

| t [min] | C-1 | C-2 | E-1 | E-2 | F-1 | F-2 |
|---|---|---|---|---|---|---|
| 2 | 79.57 | 92.62 | 99.32 | 95.38 | 90.31 | 96.05 |
| 4 | 93.67 | 99.12 | 100.25 | 96.67 | 95.86 | 99.26 |
| 6 | 99.13 | 99.74 | 99.24 | 95.80 | 98.37 | 100.16 |
| 8 | 99.87 | 99.73 | 99.47 | 96.46 | 99.10 | 1.21 |
| 10 | 99.63 | 99.55 | 100.58 | 96.47 | 98.48 | 100.92 |
| 12 | 99.80 | 99.73 | 99.42 | 96.45 | 98.55 | 101.24 |
| 15 | 99.92 | 99.56 | 98.40 | 96.19 | 99.15 | 101.22 |
| 30 | 100.27 | 99.77 | 100.24 | 96.42 | 98.74 | 101.26 |

In a few cases, in particular in the case of the hydrochlorides, after two to 4 minutes nearly 100% of the substance had passed into the artificial intestinal fluid. In other cases, however, a more intensely prolonged release occurred. An example of this is the palmitate in Experiments B-1 and B-2.

Values clearly differing from values of around 100% residual substance are to be attributed to obvious measuring errors.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A crystalline salt or co-crystal of (+)-3-[2-(dimethylamino)methyl-1-hydroxycyclohexyl]phenol or (−)-3-[2-(dimethylamino)methyl-1-hydroxycyclohexyl]phenol with an aromatic or aliphatic carboxylic acid selected from the group consisting of cinnamic acid, C₆- to C₁₀-alkanemonocarboxylic acids, C₈- to C₁₀-alkanedicarboxylic acids, C₁₅- to C₁₇-alkanemonocarboxylic acids, fumaric acid, tartaric acid, mandelic acid, hippuric acid and embonic acid.

2. A salt or co-crystal according to claim 1, wherein the carboxylic acid is cinnamic acid, and an X-ray powder diffractogram of said salt or co-crystal comprises one or more of the following reflections in each case ±0.2 in 20: 7.9; 11.4; 12.6; 14.7; 15.8; 16.1; 17.3; 18.7; 19.1; 19.9; 20.5; 21.3; 22.5; 23.4; 23.6; 29.7; 30.9.

3. A salt or co-crystal according to claim 1, wherein the carboxylic acid is palmitic acid, and an X-ray powder diffractogram of said salt or co-crystal comprises one or more of the following reflections in each case ±0.2 in 2θ: 5.3; 7.9; 8.7; 10.4; 11.6; 13.0; 13.5; 15.8; 16.5; 17.4; 17.7; 19.0; 20.1; 20.3; 20.5; 21.3; 22.1; 22.5; 23.2; 24.0.

4. A salt or co-crystal according to claim 1, wherein the carboxylic acid is octanoic acid, and an X-ray powder diffractogram of said salt or co-crystal comprises one or more of the following reflections in each case ±0.2 in 2θ: 7.6; 12.9; 16.6; 18.5; 18.8; 19.6; 20.8; 22.2; 23.7; 24.0; 31.9.

5. A salt or co-crystal according to claim 1, wherein the carboxylic acid is decanoic acid, and an X-ray powder diffractogram of said salt or co-crystal comprises one or more of the following reflections in each case ±0.2 in 2θ: 5.4; 6.1; 7.1; 8.3; 10.3; 12.6; 13.3; 14.2; 15.9; 16.8; 17.6; 18.7; 19.0; 19.8; 20.2; 20.4; 20:9; 21.5; 21.7; 22.5; 23.1; 23.8; 31.9.

6. A salt or co-crystal according to claim 1, wherein the carboxylic acid is fumaric acid, and an X-ray powder diffractogram of said salt or co-crystal comprises one or more of the following reflections in each case ±0.2 in 2θ: 7.2; 9.4; 9.5; 9.9; 13.1; 13.5; 13.9; 14.3; 14.5; 15.3; 17.8; 18.0; 18.9; 19.2; 19.9; 20.1; 20.4; 20.7; 21.2; 21.8; 22.3; 22.8; 23.3; 25.3; 25.9; 26.1; 27.4; 28.0; 28.8; 35.7.

7. A salt or co-crystal according to claim 1, wherein the carboxylic acid is sebacic acid, and an X-ray powder diffractogram of said salt or co-crystal comprises one or more of the following reflections in each case ±0.2 in 20: 7.9; 8.6; 11.7; 12.6; 13.1; 13.5; 14.2; 15.8; 16.4; 16.7; 17.1; 18.0; 18.5; 19.0; 19.5; 20.2; 20.6; 21.5; 22.5; 22.9; 23.5; 24.2; 24.6; 25.8; 26.1; 26.4; 27.5; 32.2; 32.4.

8. A process for producing a salt or co-crystal according to claim 1, said process comprising:
providing (+)-3-[2-(dimethylamino)methyl-1-hydroxycyclohexyl]phenol or (−)-3-[2-(dimethylamino)methyl-1-hydroxycyclohexyl]phenol;
providing an aromatic or aliphatic carboxylic acid selected from the group consisting of cinnamic acid, C₆- to C₁₀-alkanemonocarboxylic acids, C₈- to C₁₀-alkanedicarboxylic acids, C₁₅- to C₁₇-alkanemonocarboxylic acids, fumaric acid, tartaric acid, mandelic acid, hippuric acid and embonic acid;
adding a solvent to a mixture of 3-[2-(dimethylamino)methyl-1-hydroxycyclohexyl]phenol and the carboxylic acid, and
thereafter removing the solvent.

9. A process according to claim 8, wherein the carboxylic acid is not sebacic acid, and the solvent is dichloromethane.

10. A process according to claim 8, wherein the carboxylic acid is sebacic acid, and the solvent is tert-butyl methyl ether, acetone or isopropanol.

11. A pharmaceutical composition comprising a salt or co-crystal according to claim 1, and at least one pharmaceutical carrier or auxiliary substance.

12. A pharmaceutical composition according to claim 11, wherein the pharmaceutical composition exhibits a delayed, prolonged or extended release.

13. A method of treating a disease or condition selected from the group consisting of pain, acute pain, chronic pain, neuropathic pain, visceral pain, migraine, depression, cough, urinary incontinence, irritable bladder, diarrhea, pruritus, muscle spasms, cramps, convulsions, alcohol abuse, drug abuse, nicotine abuse, cocaine abuse, alcohol dependence, drug dependence, nicotine dependence and cocaine dependence in a subject in need of such treatment, said method comprising administering to said subject a pharmaceutically effective amount of a composition comprising a salt or co-crystal according to claim 1.

14. A method according to claim 13, wherein said condition is pain.

15. A method according to claim 14, wherein said pain is selected from the group consisting of acute pain, chronic pain, neuropathic pain and visceral pain.
